(19) United States
(12) Patent Application Publication
(10) Pub. No.: US 2023/0352154 A1
(43) Pub. Date: Nov. 2, 2023

(54) METHODS AND SYSTEMS FOR MANAGING HEALTHCARE WORKFLOWS

(71) Applicant: Neelendu Bose, Kinnelon, NJ (US)

(72) Inventor: Neelendu Bose, Kinnelon, NJ (US)

(21) Appl. No.: 18/139,823

(22) Filed: Apr. 26, 2023

Related U.S. Application Data
(60) Provisional application No. 63/335,475, filed on Apr. 27, 2022.

(51) Int. Cl. G16H 40/20

(52) U.S. Cl. CPC G16H 40/20

(57) ABSTRACT

The subject matter described herein includes methods, systems, and computer program products for managing healthcare workflows. According to one method, healthcare data associated with a healthcare service is obtained from at least one external source and analyzed. An interface is provided for viewing and managing the healthcare data and for receiving instructions from a user for performing an action. The action is then performed based on the instructions, the healthcare data, and the analysis.

### CROSS-REFERENCE TO RELATED APPLICATIONS

This application claims the benefit of priority of U.S. provisional patent application No. 63/335,475, titled “METHODS, SYSTEMS, AND COMPUTER PROGRAM PRODUCTS FOR MANAGING HEALTHCARE WORKFLOWS”, filed on Apr. 27, 2022, which is incorporated by reference herein in its entirety.

### TECHNICAL FIELD

The present invention relates to healthcare, and more specifically, to managing healthcare workflows.

### BACKGROUND

The United States (US) has a population of over 330 million people and is supported by one of the most complex healthcare systems in the world, formed by intertwining relationships between providers, payers, and patients receiving care. Moreover, the US healthcare system is in a constant state of change.

The US healthcare system does not provide universal coverage and is instead a mixed system, where publicly financed government Medicare and Medicaid health coverage coexists with privately financed market coverage (private health insurance plans). Out-of-pocket payments and market provision of coverage predominate as a means of financing and providing healthcare. As of 2019, around 50% of patients received private insurance coverage through their employer (group insurance), 6% received private insurance through health insurance marketplaces (nongroup insurance), 20% relied on Medicaid, 14% on Medicare, and 1% on other public forms of insurance (e.g., Veterans Health Administration and Military Health Service). Public and private hospitals receive payment from both public and private financing sources.

Hospitals are typically paid through a diagnostic-related group (DRG), which assigns a set payment amount for a particular condition or treatment sequence. Inpatient DRGs are widely used by the Centers for Medicare & Medicaid Services (CMS) and by many private payers as a payment scheme for hospitals. In the outpatient setting, Ambulatory Payment Classification (APC) codes are used by the hospital system for billing and reimbursement. These APC codes represent a fee-for-service style of billing, rather than the capped, cost-based style of DRGs. When billing for physicians and other clinician fees, Current Procedural Terminology (CPT) codes are used and are billed under the name of the provider rather than the hospital. CPT codes may be used in both the inpatient and outpatient settings and are indicative of a fee-for-service healthcare reimbursement structure. Private insurers pay hospitals based on DRGs, case rates, per diems, fee-for-service, and/or discounted fee-for-service schemes. On average, these payments exceed the hospital's costs of providing the underlying services.

Navigating and managing the byzantine healthcare system and enormous amount of data that can be generated and associated with a patient, procedure, or provider over months or even years can be difficult to impossible currently. Additionally, manual or semi-automated methods and systems for managing healthcare workflows are prone to error, inefficiencies, sub-optimal outcomes, or other drawbacks.

One example of a healthcare workflow, is an appeal of a health plan decision, such as a denial of a healthcare insurance claim. Regulations give patients the right to appeal decisions made by their health plans, including claim denials and rescissions. The rules issued by the Departments of Health and Human Services, Labor, and the Treasury give patients the right to two types of appeals. Patients can appeal decisions made by their health plan through the plan's internal process or patients can appeal decisions made by their health plan to an outside, independent decision-maker, referred to as an external review.

Standards established by the National Association of Insurance Commissioners (NAIL), for example, call for an external review process that includes: external review of plan decisions denying coverage for care based on medical necessity, appropriateness, health care setting, level of care, or effectiveness of a covered benefit; clear information for consumers about their right to both internal and external appeals—both in the standard plan materials, and at the tune the company denies a claim; expedited access to external review in some cases including emergency situations, or cases where their health plan did not follow the rules in the internal appeal; health plans must pay the cost of the external appeal under State law, and States may not require consumers to pay more than a nominal fee; review by an independent body assigned by the State. The State must also ensure that the reviewers meet certain standards, keep written records, and are not affected by conflicts of interest emergency processes for urgent claims and a process for experimental or investigational treatment; and final decisions must be binding so, if the consumer wins, the health plan is expected to pay for the benefit that was previously denied.

As may be appreciated from these examples, current methods and systems for managing healthcare workflows are prone to errors, inefficiencies, sub-optimal outcomes, and other drawbacks.

Accordingly, a need exists for improved systems and methods for managing healthcare workflows that address these deficiencies.

### SUMMARY

The subject matter described herein includes methods, systems, and computer program products for managing healthcare workflows. According to one method, healthcare data associated with a healthcare service is obtained from at least one external source and analyzed. An interface is provided for viewing and managing the healthcare data and for receiving instructions from a user for performing an action. The action is then performed based on the instructions, the healthcare data, and the analysis.

A system for managing healthcare workflows includes data storage and a server, the server including: an input module, an analysis module, an interface module, and an application module. The input module is configured to receive the healthcare data from at least one external source. The data storage is configured to store the healthcare data. The analysis module is configured to analyze the healthcare data stored in the data storage. The interface module is configured to provide an interface for viewing and managing the healthcare data and for receiving instructions from a user for performing an action. The application module is configured to perform the action based on the instructions, the healthcare data, and the analysis.

A system for managing healthcare workflows as a service (SaaS) includes: a computer communicatively coupled to a network, at least one SaaS provider, and at least one SaaS user. The computer is configured to obtain healthcare data associated with a healthcare service from at least one external source and to analyze the healthcare data. The computer is also configured to provide an interface for viewing and managing the healthcare data and for receiving instructions from a user for performing an action. The computer is also configured to perform the action based on the instructions, the healthcare data, and the analysis.

A computer-implemented method comprising instructions stored on a non-transitory computer-readable storage medium and executed on a computing device provided with a hardware processor and a memory for managing healthcare workflows via Software as a Service (SaaS). The method includes obtaining healthcare data associated with a healthcare service from at least one external source and analyzing the healthcare data. The method also includes providing an interface for viewing and managing the healthcare data and receiving instructions from a user for performing an action. The method also includes performing the action based on the instructions, the healthcare data, and the analysis.

### BRIEF DESCRIPTION OF THE DRAWINGS

FIG. 1 is a flow diagram illustrating exemplary steps for managing healthcare workflows according to an embodiment of the subject matter described herein.

FIG. 2 is a system diagram illustrating exemplary functional components for managing healthcare workflows according to an embodiment of the subject matter described herein.

FIG. 3 is a diagram illustrating an exemplary data flow for managing healthcare workflows according to an embodiment of the subject matter described herein.

FIG. 4 is a diagram illustrating an exemplary user interface for uploading data according to an embodiment of the subject matter described herein.

FIGS. 5A, 5B, and 5C are diagrams illustrating an exemplary user interface for managing healthcare workflows according to an embodiment of the subject matter described herein.

FIG. 6 is a diagram illustrating an exemplary activity log user interface for managing healthcare workflows according to an embodiment of the subject matter described herein.

FIG. 7 is a diagram illustrating an exemplary task manager user interface for managing healthcare workflows according to an embodiment of the subject matter described herein.

FIGS. 8A and 8B are diagrams illustrating exemplary table user interface for managing healthcare workflows according to an embodiment of the subject matter described herein.

FIGS. 9A, 9B, and 9C are diagrams illustrating an exemplary user interface for filing an appeal as part of managing healthcare workflows according to an embodiment of the subject matter described herein.

FIGS. 10A, 10B, and 10C are diagrams illustrating an exemplary user interface that may be displayed after selecting the Appeals tab in FIG. 9B for filing an appeal as part of managing healthcare workflows according to an embodiment of the subject matter described herein.

FIGS. 11A, 11B, and 11C are diagrams illustrating an exemplary user interface that may be displayed after selecting Appeal Level 1 in FIG. 10B for filing an appeal as part of managing healthcare workflows according to an embodiment of the subject matter described herein.

FIGS. 12A, 12B, and 12C are diagrams illustrating an exemplary user interface that may be displayed after reviewing and verifying the information shown in FIG. 11B for selecting a method for sending the appeal as part of managing healthcare workflows according to an embodiment of the subject matter described herein.

FIGS. 13A and 13B are diagrams illustrating an exemplary draft appeal letter as part of managing healthcare workflows according to an embodiment of the subject matter described herein.

FIGS. 14A, 14B, and 14C are diagrams illustrating an exemplary phone call scheduling user interface for filing an appeal as part of managing healthcare workflows according to an embodiment of the subject matter described herein.

FIG. 15 is a diagram illustrating an exemplary Practice Summary page user interface for selecting a Provider for rejecting a negotiation as part of managing healthcare workflows according to an embodiment of the subject matter described herein.

FIGS. 16A and 16B are diagrams illustrating an exemplary patient search user interface for rejecting a negotiation as part of managing healthcare workflows according to an embodiment of the subject matter described herein.

FIGS. 17A and 17B are diagrams illustrating an exemplary user interface if claims are not already present in the database for rejecting a negotiation as part of managing healthcare workflows according to an embodiment of the subject matter described herein.

FIGS. 18A and 18B are diagrams illustrating an exemplary daily log user interface that may be displayed after the user interface of FIGS. 17A-17B for rejecting a negotiation as part of managing healthcare workflows according to an embodiment of the subject matter described herein.

FIGS. 19A and 19B are diagrams illustrating an exemplary manual input user interface that may be displayed after the user interface of FIGS. 18A-18B for rejecting a negotiation as part of managing healthcare workflows according to an embodiment of the subject matter described herein.

FIGS. 20A and 20B are diagrams illustrating an exemplary user interface that may be displayed after clicking Submit in FIGS. 19A-19B for rejecting a negotiation as part of managing healthcare workflows according to an embodiment of the subject matter described herein.

FIGS. 21A and 21B are diagrams illustrating an exemplary user interface that may be displayed after clicking OK in FIGS. 20A-20B for rejecting a negotiation as part of managing healthcare workflows according to an embodiment of the subject matter described herein.

FIG. 22 is a diagram illustrating an exemplary pdf of the faxed negotiation and tools for editing the faxed negotiation pdf rejecting a negotiation as part of managing healthcare workflows according to an embodiment of the subject matter described herein.

FIGS. 23A and 23B are diagrams illustrating an exemplary pdf of the faxed negotiation and tools for editing the faxed negotiation pdf rejecting a negotiation as part of managing healthcare workflows according to an embodiment of the subject matter described herein.

FIGS. 24A and 24B are diagrams illustrating an exemplary pdf of the faxed negotiation and tools for signing the faxed negotiation pdf rejecting a negotiation as part of managing healthcare workflows according to an embodiment of the subject matter described herein.

FIGS. 25A, 25B, and 25C are diagrams illustrating an exemplary user interface for entering negotiation details and uploading the negotiation file for rejecting a negotiation as part of managing healthcare workflows according to an embodiment of the subject matter described herein.

FIG. 26 is a diagram illustrating an exemplary user interface for dragging and dropping an edited negotiation for rejecting a negotiation as part of managing healthcare workflows according to an embodiment of the subject matter described herein.

FIGS. 27A, and 27B are diagrams illustrating an exemplary user interface that may be displayed after clicking Submit in FIG. 26 for rejecting a negotiation as part of managing healthcare workflows according to an embodiment of the subject matter described herein.

FIG. 28 is a diagram illustrating an exemplary user interface for rejecting a negotiation as part of managing healthcare workflows according to an embodiment of the subject matter described herein.

FIG. 29 is a diagram illustrating an exemplary user interface for creating and previewing a negotiation rejection for rejecting a negotiation as part of managing healthcare workflows according to an embodiment of the subject matter described herein.

FIGS. 30A and 30B are diagrams illustrating an exemplary user interface for editing a negotiation letter for rejecting a negotiation as part of managing healthcare workflows according to an embodiment of the subject matter described herein.

FIGS. 31A, 31B, 31C, 31D, 31E, and 31F are diagrams illustrating portions of an exemplary user interface for documenting phone calls as part of managing healthcare workflows according to an embodiment of the subject matter described herein.

FIG. 32 is a diagram illustrating an exemplary Software-as-a-Service (SaaS) platform suitable for managing healthcare workflows according to an embodiment of the subject matter described herein.

### DETAILED DESCRIPTION

The following description and figures are illustrative and are not to be construed as limiting. Numerous specific details are described to provide a thorough understanding of the disclosure. In certain instances, however, well-known or conventional details are not described in order to avoid obscuring the description. References to “one embodiment” or “an embodiment” in the present disclosure may be (but are not necessarily) references to the same embodiment, and such references mean at least one of the embodiments.

Reference in this specification to “one embodiment” or “an embodiment” means that a particular feature, structure, or characteristic described in connection with the embodiment is included in at least one embodiment of the disclosure. Multiple appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

In contrast to conventional methods and systems which provide little to no guidance for successfully navigating the appeal process, the present disclosure assists users and makes the appeal process as simple as possible. Providers can obtain remedies, or full claim payments, through the system of working all levels of appeal as needed. When many claims are involved, further gains can be obtained, and users experience a decrease in claim denials overall. The system contains built-in expert intelligence about the appeal process and automatically generates customized appeal documents, including citing landmark cases, and references specific laws and regulations to further promote the proper payment of claims.

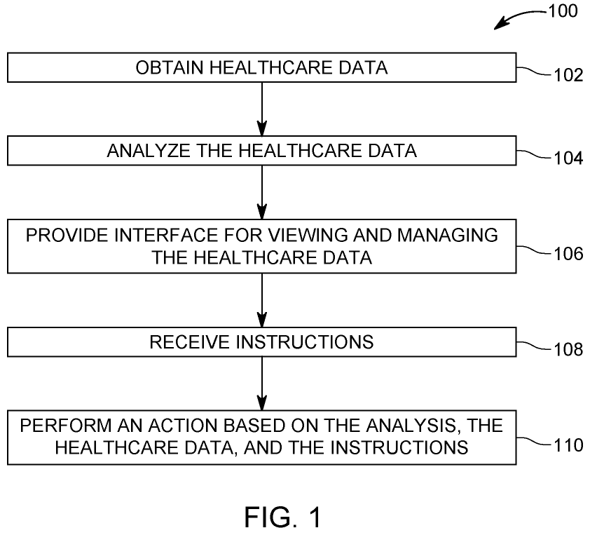

FIG. 1 is a flow diagram illustrating exemplary steps for managing healthcare workflows according to an embodiment of the subject matter described herein.

At step 100, healthcare data associated with a healthcare service is obtained from at least one external source. In one embodiment, the healthcare data includes one or more of: an 835, an 837, a 277CA, and a portable document format (PDF) file. As will be described in greater detail below, Health Level Seven (HL7) is a standard for exchanging information between medical information systems.

In HL7, the 837 file includes insurance claim data for one or more claims from the hospital to the payor, such as details of patients' treatment, including medical services provided, cost of treatment, and the actual claim amount.

The 835 is an electronic transaction sent from insurers to the healthcare provider that provides claim payment information and documents the EFT (electronic funds transfer).

The 277CA healthcare claim acknowledgment provides a claim-level acknowledgment of all claims received in the payer's pre-processing system before submitting claims into an adjudication system.

The PDF file may be associated with a variety of different documents or formats. For example, a document associated with negotiation may be conducted via a fax formatted as a Vonage fax. Alternatively, the document may be associated with negotiation may be formatted as an eFax. In other embodiments, the PDF may be associated with a document that is part of an appeal, such as a response by one or more of the parties involved.

The PDF may be formatted as an image or as a machine-readable text format. Therefore, the method may include using optical character recognition (OCR) on the PDF to convert the image of text into the machine-readable text format if necessary.

At step 102, the healthcare data is analyzed. Analyzing the healthcare data may include extracting key data from the received healthcare data. The key data extracted from the received healthcare data may include: date of birth, invoice number, claim data, identifier number, patient name, phone number, fax number, expiration data, provider name, billed amount, proposed amount, keywords, paid amount, co-insurance, deductible, claim adjustment reason code (CARC), remittance advice remark codes (RARC), place of service (POS), elective versus emergency room (ER) indication, insurance (INS) claim number, and authorization.

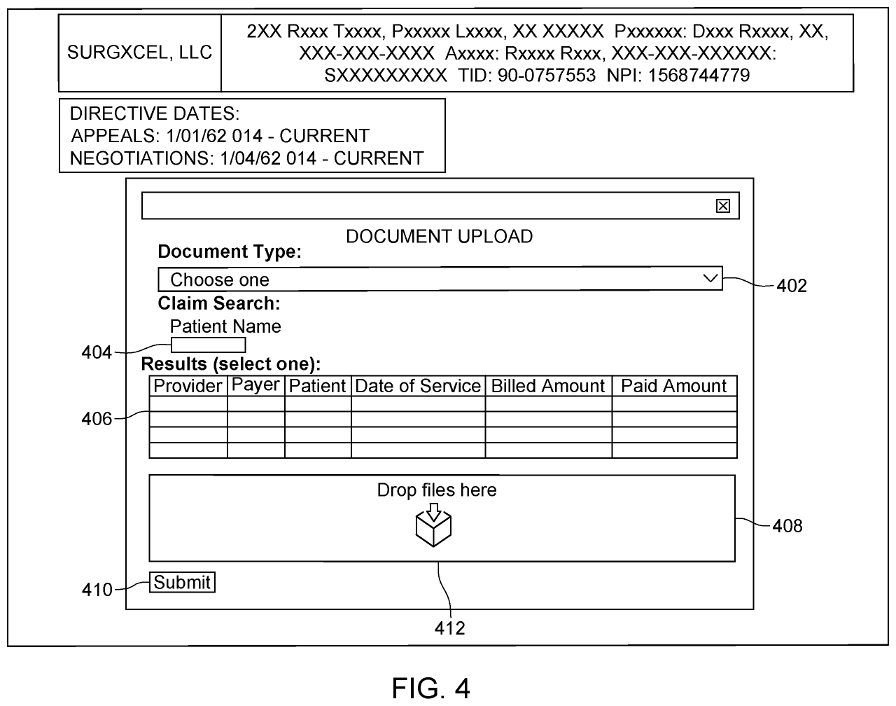

At step 104, an interface is provided for viewing and managing the healthcare data. In one embodiment, providing the interface for viewing and managing the healthcare data includes displaying a graphical user interface (GUI) to a user. Details and example screenshots illustrating a GUI for managing healthcare workflows will be described in greater detail below with respect to FIGS. 4-8.

At step 106, instructions for performing an action are received from a user. For example, the user may generate one or more documents associated with an appeal, generate one or more documents associated with a negotiation, generate a phone script, view and revise an up-to-date list of tasks in a task manager, view a communication log, generate reports, or generating a client invoice.

In other embodiments, such as one where the user is an external client such as a provider and the interface provided is a “Provider Portal” or “Provider Dashboard”, the user may check eligibility, receive documents uploaded by another user, provide a communication log, generate a status report, or check compliance.

At step 108, the action is performed based on the instructions, the healthcare data, and the analysis. For example, the action may include: generating one or more documents associated with an appeal, generating one or more documents associated with a negotiation, generating a phone script, updating a task manager, updating a communication log, generating a report, generating client invoicing, checking eligibility, receiving documents uploaded by another user, providing a communication log, generating a status report, and checking compliance.

It is appreciated that some actions may include viewing or managing data internal to the system, such as calendar, tasks, and reports, while other actions may include sending or receiving data from external sources. As previously mentioned, receiving data from external sources may involve various data aggregation, formatting, translation, or normalization procedures.

Actions that involve sending data to external sources may often include an intermediary step of generating the data to be sent. For example, as part of an appeal process, data may be received requiring a response in the form of an appeal letter. The system may automatically generate the appeal letter for the user and the appeal letter can be automatically transmitted and tracked by the system.

It is appreciated that the appeal letter generated by the system may be based on the healthcare data received (e.g., claim denial), the instructions received from the user (file an appeal), and an analysis of the data. This analysis can include populating one of a plurality of template appeal letters based on the data. For example, a first appeal letter template may be selected by the analysis module of the system because it most closely aligns with the data (e.g., different healthcare providers or different States may require different language in their appeal letters). This first appeal letter template may be one of several templates available for selection.

Using the selected template, the system may customize the appeal letter for the specific user or appeal. For example, citations to certain court cases or references to specific laws and regulations may be inserted into the appeal letter, where the customized citations and references are determined by the analysis module to further promote the proper payment of claims. This determination may be based on an algorithm or other hard-coded intelligence that implements knowledge of experts in navigating the appeal process. The language customizations and the appeal letter templates may also be based on expert human knowledge of the appeal process. Alternatively, this determination may be based on machine learning or other models that adjust language based on evidence of successful appeals.

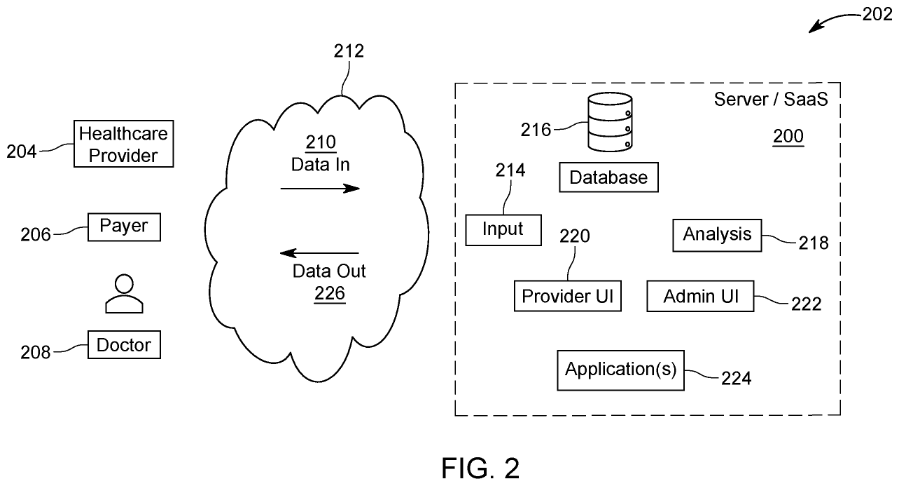

FIG. 2 is a system diagram illustrating exemplary functional components for managing healthcare workflows according to an embodiment of the subject matter described herein.

The system 200 includes functions executed in software by a computing device connected to a network. The computing device can include one or more servers that can be located locally or remotely from users.

The system disclosed herein may be implemented as a client/server type architecture but may also be implemented using other architectures, such as cloud computing, software as a service model (SaaS), a mainframe/terminal model, a stand-alone computer model, a plurality of non-transitory lines of code on a computer readable medium that can be loaded onto a computer system, a plurality of non-transitory lines of code downloadable to a computer, and the like. In one embodiment, the system 200 may be hosted in the cloud as Software as a Service (SaaS).

The system may be implemented as one or more computing devices that connect to, communicate with and/or exchange data over a link that interact with each other. Each computing device may be a processing unit-based device with sufficient processing power, memory/storage and connectivity/communications capabilities to connect to and interact with the system. For example, each computing device may be an Apple iPhone or iPad product, a Blackberry or Nokia product, a mobile product that executes the Android operating system, a personal computer, a tablet computer, a laptop computer and the like and the system is not limited to operate with any particular computing device. The link may be any wired or wireless communications link that allows the one or more computing devices and the system to communicate with each other. In one example, the link may be a combination of wireless digital data networks that connect to the computing devices and the Internet. The system may be implemented as one or more server computers (all located at one geographic location or in disparate locations) that execute a plurality of lines of non-transitory computer code to implement the functions and operations of the system as described herein. Alternatively, the system may be implemented as a hardware unit in which the functions and operations of the back-end system are programmed into a hardware system. In one implementation, the one or more server computers may use Intel® processors, run the Linux operating system, and execute Java, Ruby, Regular Expression, Flex 4.0, SQL etc.

In some embodiments, each computing device may further comprise a display and a browser application so that the display can display information generated by the system. The browser application may be a plurality of non-transitory lines of computer code executed by a processing unit of the computing device. Each computing device may also have the usual components of a computing device such as one or more processing units, memory, permanent storage, wireless/wired communication circuitry, an operating system, etc.

The system may further comprise a server (that may be software based or hardware based) that allows each computing device to connect to and interact with the system such as sending information and receiving information from the computing devices that is executed by one or more processing units. The system may further comprise software- or hardware-based modules and database(s) for processing and storing content associated with the system, metadata generated by the system for each piece of content, user preferences, and the like.

In one embodiment, the system includes one or more processors, server, clients, data storage devices, and non-transitory computer readable instructions that, when executed by a processor, cause a device to perform one or more functions. It is appreciated that the functions described herein may be performed by a single device or may be distributed across multiple devices.

When a user interacts with the system, the user may use a frontend client application. The client application may include a graphical user interface that allows the user to select one or more digital files. The client application may communicate with a backend cloud component using an application programming interface (API) comprising a set of definitions and protocols for building and integrating application software. As used herein, an API is a connection between computers or between computer programs that is a type of software interface, offering a service to other pieces of software. A document or standard that describes how to build or use such a connection or interface is called an API specification. A computer system that meets this standard is said to implement or expose an API. The term API may refer either to the specification or to the implementation.

Software-as-a-service (SaaS) is a software licensing and delivery model in which software is licensed on a subscription basis and is centrally hosted. SaaS is typically accessed by users using a thin client, e.g., via a web browser. SaaS is considered part of the nomenclature of cloud computing.

Many SaaS solutions are based on a multitenant architecture. With this model, a single version of the application, with a single configuration (hardware, network, operating system), is used for all customers (“tenants”). To support scalability, the application is installed on multiple machines (called horizontal scaling). The term “software multitenancy” refers to a software architecture in which a single instance of software runs on a server and serves multiple tenants. Systems designed in such manner are often called shared (in contrast to dedicated or isolated). A tenant is a group of users who share a common access with specific privileges to the software instance. With a multitenant architecture, a software application is designed to provide every tenant a dedicated share of the instance—including its data, configuration, user management, tenant individual functionality and non-functional properties.

The backend cloud component described herein may also be referred to as a SaaS component. One or more tenants may communicate with the SaaS component via a communications network, such as the Internet. The SaaS component may be logically divided into one or more layers, each layer providing separate functionality and being capable of communicating with one or more other layers.

Cloud storage may store or manage information using a public or private cloud. Cloud storage is a model of computer data storage in which the digital data is stored in logical pools. The physical storage spans multiple servers (sometimes in multiple locations), and the physical environment is typically owned and managed by a hosting company. Cloud storage providers are responsible for keeping the data available and accessible, and the physical environment protected and running. People and/or organizations buy or lease storage capacity from the providers to store user, organization, or application data. Cloud storage services may be accessed through a co-located cloud computing service, a web service API, or by applications that utilize the API.

In an exemplary SaaS model, the functionality disclosed herein can be logically divided into layers. Layers may be ordered from least to greatest abstraction of underlying physical resources. Layers may also be divided into groups based on common features for simplicity when referring or billing functions associated with each group.

Infrastructure may include a storage function, a networking function, a server function, and a virtualization function. Infrastructure functions may be bundled together and provided to one or more tenants as a service, referred to as Infrastructure-as-a-Service (IaaS). IaaS is made up of a collection of physical and virtualized resources that provide consumers with the basic building blocks needed to run applications and workloads in the cloud.

The storage function provides storage of data without requiring the user or tenant to be aware of how this data storage is managed. Three types of cloud storage that may be provided by the storage function are: block storage, file storage, and object storage. Object storage is the most common mode of storage in the cloud because it is highly distributed (and thus resilient), data can be accessed easily over HTTP, and performance scales linearly as the storage grows.

The networking function in the cloud is a form of software defined networking in which traditional networking hardware, such as routers and switches, are made available programmatically, typically through APIs.

The server function refers to various physical hardware resources associated with executing computer-readable code that is not otherwise part of the virtualized network resources in the networking function or the storage function. IaaS providers manage large data centers, typically around the world, that contain the servers powering the various layers of abstraction on top of them and that are made available to end users. In most IaaS models, end users do not interact directly with the physical infrastructure (e.g., memory, motherboard, CPU), but it is provided as a service to them.

The virtualization function provides virtualization of underlying resources via one or more virtual machines (VMs). Virtualization relies on software to simulate hardware functionality and create a virtual computer system. A virtual computer system is known as a “virtual machine” (VM): a tightly isolated software container with an operating system and application inside. Each self-contained VM is completely independent. Putting multiple VMs on a single computer enables several operating systems and applications to run on just one physical server, or “host.” A thin layer of software called a “hypervisor” decouples the virtual machines from the host and dynamically allocates computing resources to each virtual machine as needed. Providers manage hypervisors and end users can then programmatically provision virtual “instances” with desired amounts of compute and memory (and sometimes storage). Most providers offer both CPUs and GPUs for different types of workloads.

The platform may include an operating system, middleware, and runtime. The infrastructure functions and the platform functions may be bundled together and provided to one or more tenants as a service, referred to as Platform-as-a-Service (PaaS). In the Platform-as-a-Service (PaaS) model, developers rent everything needed to build an application, relying on a cloud provider for development tools, infrastructure, and operating systems.

The operating system function refers to a PaaS vendor providing and maintaining the operating system that developers use, and the application runs on. For example, Windows and Linux operating systems may be installed in virtual machines and Windows or Linux applications may be run within the operating system.

The middleware is software that sits in between user-facing applications and the machine's operating system. For example, middleware may allow software to access input from the keyboard and mouse. Middleware is necessary for running an application but end users don't interact with it. Relatedly, the middleware function may also include tools that are necessary for software development, such as a source code editor, a debugger, and a compiler. These tools may be offered together as a framework.

The runtime is software code that implements portions of a programming language's execution model. A runtime system or runtime environment implements portions of an execution model. Most programming languages have some form of runtime system that provides an environment in which programs run. This environment may address issues including the management of application memory, how the program accesses variables, mechanisms for passing parameters between procedures, interfacing with the operating system, and otherwise. The compiler makes assumptions depending on the specific runtime system to generate correct code. Typically, the runtime system will have some responsibility for setting up and managing the stack and heap, and may include features such as garbage collection, threads or other dynamic features built into the language.

The software includes applications and data functions. The infrastructure, platform, and software may be bundled together and provided to one or more tenants as a service, referred to as Software-as-a-Service (SaaS). Applications and data refer to the application and associated data created and managed by the user. For example, an application programmed by the user for providing functionality disclosed herein and may be exposed to the end user via a front-end interface such as a web browser or dedicated front-end client application. Neither the front-end user nor the back-end developer is required to manage or maintain services provided by the platform and the infrastructure. This contrasts with on-site hosting of the same functionality.

The system 200 may receive data from one or more external data sources 202. These external data sources 202 may include, for example, healthcare provider 204, payer 206, doctor 208.

Data 210 may be communicated to server 200 via one or more networks 212, such as the Internet.

Data 210 may be received by an input module 214 and stored in a database 216.

In one embodiment, the healthcare data stored in database 216 includes one or more of: an 835, an 837, a 277CA, and a portable document format (PDF) file. As will be described in greater detail below, Health Level Seven (HL7) is a standard for exchanging information between medical information systems.

Database 216 may be accessed by one or more modules of the system 200. For example, an administrative user interface (UI) module may access database 216 for administrative uses such as error logs, software updates, and adding or removing users.

Provider UI module 220 may access database 216 for displaying healthcare data to a user via a GUI such as a dashboard. For example, the provider UI module 220 may generate and display a provider/external client dashboard. Via the provider/external client dashboard, a provider or external client may perform various actions on the data in database 216, also referred to as applications. These provider or external client applications may include an eligibility checker, a document uploader, a communications log, status reports, and compliance.

In addition to the unified portal for viewing data stored in database 216 that is part of a healthcare workflow, the system 200 may also include an applications module 224 for performing an action based on received instructions and the data stored in the database 216.

In addition to the provider or external client application mentioned above, a user dashboard may allow other users to perform various actions, also referred to as applications. These applications may include drafting an appeal letter, drafting a negotiation letter, generating a phone script, viewing a task manager, communication reports, and client invoicing.

The application module 224 may operate in conjunction with an analysis module 228 that is configured to analyze the data in database 216. Analysis module 228 may be configured to analyze the received raw healthcare data to make a determination and/or generate new or additional data which may also be stored in database 216. For example, the analysis module 228 may analyze multiple client invoices to determine that an appeal is warranted. Further, the analysis module 228 may determine various aspects of the appeal that are not included in the healthcare data, such as dates, jurisdictions, case law, etc. The appeal letter may be automatically generated, if instructed to do so based on user instructions, based on these determinations and the underlying healthcare data.

Machine learning (ML) is the use of computer algorithms that can improve automatically through experience and using data. Machine learning algorithms build a model based on sample data, also known as training data, to make predictions or decisions without being explicitly programmed to do so. Machine learning algorithms are used where it is unfeasible to develop conventional algorithms to perform the needed tasks.

In certain embodiments, instead of, or in addition to, performing the functions described herein manually, the system may perform some or all the functions using machine learning or artificial intelligence. Thus, in certain embodiments, machine learning-enabled software relies on unsupervised and/or supervised learning processes to perform the functions described herein in place of a human user.

Machine learning may include identifying one or more data sources and extracting data from the identified data sources. Instead of or in addition to transforming the data into a rigid, structured format, in which certain metadata or other information associated with the data and/or the data sources may be lost, incorrect transformations may be made, or the like, machine learning-based software may load the data in an unstructured format and automatically determine relationships between the data. Machine learning-based software may identify relationships between data in an unstructured format, assemble the data into a structured format, evaluate the correctness of the identified relationships and assembled data, and/or provide machine learning functions to a user based on the extracted and loaded data, and/or evaluate the predictive performance of the machine learning functions (e.g., “learn” from the data).

In certain embodiments, machine learning-based software assembles data into an organized format using one or more unsupervised learning techniques. Unsupervised learning techniques can identify relationships between data elements in an unstructured format.

In certain embodiments, machine learning-based software can use the organized data derived from the unsupervised learning techniques in supervised learning methods to respond to analysis requests and to provide machine learning results, such as a classification, a confidence metric, an inferred function, a regression function, an answer, a prediction, a recognized pattern, a rule, a recommendation, or other results. Supervised machine learning, as used herein, comprises one or more modules, computer executable program code, logic hardware, and/or other entities configured to learn from or train on input data, and to apply the learning or training to provide results or analysis for subsequent data.

Machine learning-based software may include a model generator, a training data module, a model processor, a model memory, and a communication device. Machine learning-based software may be configured to create prediction models based on the training data. In some embodiments, machine learning-based software may generate decision trees. For example, machine learning-based software may generate nodes, splits, and branches in a decision tree. Machine learning-based software may also calculate coefficients and hyper parameters of a decision tree based on the training data set. In other embodiments, machine learning-based software may use Bayesian algorithms or clustering algorithms to generate predicting models. In yet other embodiments, machine learning-based software may use association rule mining, artificial neural networks, and/or deep learning algorithms to develop models. In some embodiments, to improve the efficiency of the model generation, machine learning-based software may utilize hardware optimized for machine learning functions, such as an FPGA.

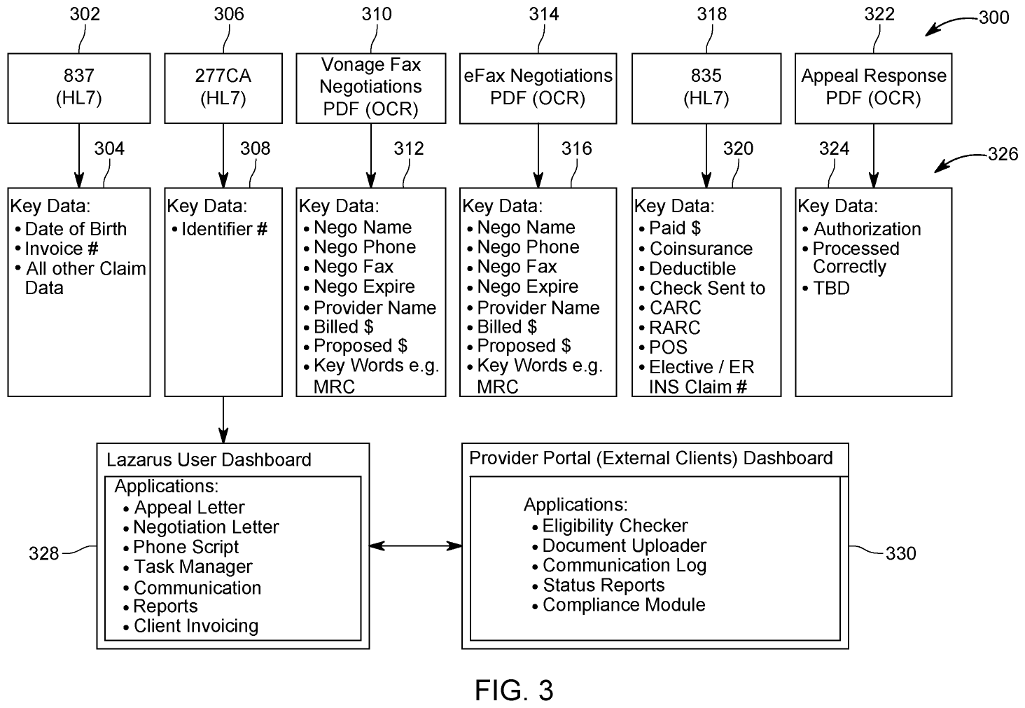

FIG. 3 is a diagram illustrating an exemplary data flow for managing healthcare workflows according to an embodiment of the subject matter described herein.

Exemplary data sources 300 include: an 837 (HL7) file 302, a 277CA (HL7) file 306, a fax negotiation (pdf) file 310 or 314, an 835 (HL7) 318 file, and/or an appeal response (pdf) file 322.

Health Level Seven (HL7) is a standard for exchanging information between medical information systems. It is widely deployed and covers the exchange of information in several functional domains. It is very important and crucial to achieve interoperability in healthcare and it is therefore a cornerstone to implement the Electronic Health Record (EHR). EHR improves healthcare decisions by allowing access to the patient's relevant clinical information at the decision-making point. EHR is a distributed system that results from the interactions and cooperation of various independent information systems to achieve a specific healthcare process.

Several versions of the HL7 standard exist. HL7 v2 is largely implemented and deployed in almost every healthcare hospital or clinic. HL7 v3 is adopted by many governments and agencies as a standard required for EHR and is used in many of the IHE integration profiles. HL7 also has a new framework, the Fast Healthcare Interoperability Resources (FHIR), which combines features of HL7 v2 and HL7 v3 with technologies such as the Representational State Transfer (REST) architecture to facilitate implementation. All versions of HL7 may co-exist and it may be common for several versions of the standard to be deployed simultaneously at the same institution.

Recovering healthcare claims from insurance providers is a convoluted process. The claims process revolves around two different files, 835s, and 837s, which may generally be understood as the bill and the receipt for a healthcare procedure.

The 837 file is a Health Insurance Portability and Accountability Act of 1996 (HIPAA) form utilized by healthcare organizations and medical providers to communicate healthcare claims. Also known as EDIs, they are essentially electronic files that contain information about an electronic claim. They are “electronic” because the file is submitted to an insurance provider in lieu of a paper claim.

Electronic Data Interchange (EDI) is the electronic interchange of business information using a standardized format; a process which allows one company to send information to another company electronically rather than with paper.

The 837 file includes insurance claim data, however, 837 files may contain not just one claim but multiple claims from the hospital to the payor. The 837s will include information that details aspects of patients' treatment, including medical services provided, cost of treatment, and additional adjustments. Finally, the 837s will consist of the actual claim amount.

An 835 is also known as an Electronic Remittance Advice (ERA). It is the electronic transaction that provides claim payment information and documents the EFT (electronic funds transfer). An 835 is sent from insurers to the healthcare provider. Similar to an 837, they also provide information about the healthcare services being paid for. This includes data like what medical treatment is being paid for and if it has been reduced or changed in the time between when the 835 remittance file was sent out. Additionally, the 835 includes insurance information about deductibles, co-pay amounts, splitting of healthcare claims, co-insurers, and bundling. From the 835 file 318, key data 320 may be obtained. Key data 320 may include: paid amount, co-insurance, deductible, claim adjustment reason code (CARC), remittance advice remark codes (RARC), place of service (POS), elective versus emergency room (ER) indication, insurance (INS) claim number.

If the 837 is analogous to the bill, then the 835 is analogous to the receipt of the bill. For example, hospitals send healthcare claims to insurers to recoup revenue, and then sometime later, insurance providers will electronically deposit money in the bank account and send a record of that transaction as an 835 file.

Healthcare claims may begin when a healthcare provider seeks to claim monetary compensation from an insurer based on a patient contract. The healthcare organization will send an EDI, an 837 file. When a hospital sends an EDI (the electronic record of a claim) to an insurance provider, the insurer doesn't immediately send a remittance payment back. It takes weeks or months for that payment to be deposited directly into a hospital's bank account via an electronic funds transfer (an EFT). However, that final payment, recorded through an 835 ERA, doesn't always match the original 837. From the 837 file 302, key data 304 may be obtained. Key data 304 may include: date of birth, invoice number, and all other claim data.

While the EFT is often accurate, the deposit information included in the 835 is often inaccurate because of adjustments to pricing made by the healthcare organization or the payor. To complicate things further, the deposit is usually large and includes hundreds or even thousands of different contract payments. The record does not always reflect that individually.

The EDI 277CA healthcare claim acknowledgment provides a claim-level acknowledgment of all claims received in the payer's pre-processing system before submitting claims into an adjudication system. It is created in receipt of an incoming EDI 837 5010 claim submission transaction. The 277CA offers a common report interface to payers and providers. The 277CA is designed to give clearinghouses and payers a standardized reporting mechanism for 5010. The 277CA is not required by HIPAA.

The general acknowledgment workflow would be that the provider sends an EDI 837 to the payer. The payer or clearinghouse returns an EDI 999 to acknowledge receipt of the 837. The payer or clearinghouse may send the 277CA. If the 837 is rejected, the payer may next send EDI 275.

The 277CA reports on whether pre-adjudication validation found the claim acceptable for adjudication. The 277CA Health Care Claim Acknowledgment includes basic file information: Submission status, Submission date, Claims submitted, Claims rejected, Claims accepted, Reasons for claim rejections.

The 277CA reports status of either accepted or rejected in the STC Segments but does not have the functionality to report ‘warnings’ that did not lead to a rejected status. The 277CA also cannot report syntax issues. The 277CA can, however, reject a claim for the syntax issues previously identified in the 999 that was accepted with error(s). From the 277CA file, key data 308 may be obtained. Key data 308 may include an identifier number.

The STC segment is a key element for providing the status notification. The STC segment consists of Claim Status Categories, Claim Status Codes and monetary amounts. Some example codes are shown in Table 1 below:

| | | |
|---|---|---|
| A0 | Acknowledgment | The claim has been forwarded to another entity, |
| | Forwarded | such as a repricing service. |
| A1 | Acknowledgment | The claim has been received. |
| | Receipt | |
| A2 | Acknowledgment | The claim has been accepted into the |
| | Accepted | adjudication system. |
| A3 | Acknowledgment | The claim has been rejected and has not been |
| | Returned as | entered into the adjudication system. |
| | Unable to Process | |
| A5 | Acknowledgment | The claim has been split upon acceptance into |
| | Split Claim | the adjudication system. |
| A6 | Acknowledgment | The claim is missing information specified in |
| | Rejected for | status details and has been rejected. |
| | Missing Information | |
| A7 | Acknowledgment | The claim has invalid information as specified |
| | Rejected for | in the status details and has been rejected. |
| | Invalid Information | |
| A8 | Acknowledgment | |
| | Rejected for | |
| | Relational Filed | |
| | in Error | |

The data sources may also include negotiation documents. Here, two examples of negotiation documents are shown.

Vonage fax negotiations 310 may be received as a pdf file. The pdf file may either be optical character recognition (OCR)d by the sender or, alternatively, may be OCRd by the system 200 once received. From the OCRd pdf, key data 312 may be obtained. Key data 312 may include: negotiation name, negotiation phone number, negotiation fax number, negotiation expiration date, provider name, billed amount, proposed amount, and any keywords.

eFax negotiations 314 may also be a pdf file received electronically. From the OCRd pdf, key data 316 may be obtained. Key data 316 may include: negotiation name, negotiation phone number, negotiation fax number, negotiation expiration date, provider name, billed amount, proposed amount, and any keywords.

Appeal response 322 may also be received electronically as a pdf file. Key data 324 that may be obtained from the appeal response pdf may include: authorization information and an indication whether the appeal was processed correctly.

As mentioned previously, Provider UI module 220 may access database 216 for displaying healthcare data 300 to a user via a GUI such as dashboard 328 or dashboard 330. The provider/external client dashboard 330 may allow the user to perform various actions on the data in database 216, also referred to as applications, which may include an eligibility checker, a document uploader, a communications log, status reports, and compliance.

In addition to the provider or external client dashboard 330, a user dashboard 328 may allow other users to perform various actions, also referred to as applications. These applications may include drafting an appeal letter, drafting a negotiation letter, generating a phone script, viewing a task manager, communication reports, and client invoicing.

FIG. 4 is a diagram illustrating an exemplary user interface for uploading data to a system for managing healthcare workflows according to an embodiment of the subject matter described herein.

Referring to FIG. 4, a graphical user interface (GUI) 400 is shown for uploading a document to system 200 by an external client, such as a provider. As such, GUI 400 may also be referred to as provider portal 400. UI element 402 may include a drop-down box that provides a list of options for selecting a document type. Options include: appeal response, EOB, refund request, negotiation, and bulk upload. When bulk upload is selected, it is not required to attach to a specific claim. Those uploads can be saved to a claim via other means.

UI element 404 allows the user to search ERAs (835s) for claims by patient name.

UI element 406 allows the user to display the search results. Here, codes function to choose the correct claim and to upload the file to that claim. UI element 404 may include several columns such as: provider, payer, patient, date of service, billed amount, and paid amount.

UI element 408 allows the user to drag and drop one or more files to be uploaded to the system 200. File names are assigned according to specification upon being uploaded. For example, in one embodiment, file naming rules may include:

Date format:YYYY-MM-DD
Document Type Abbreviations:
Appeal Response = AR
EOB = EOB
Refund Request = RR
Negotiation = NEG
Bulk Upload = BULK

For example:

```
  SURGXCEL1017_2021-01-31_DOE_JOHN_2020-10-15_NEG
  SITE ID _ DATE OF UPLOAD _ PATIENTLASTNAME _ PATIENTFIRSTNAME _{—}
DATEOFSERVICE _ DOCUMENT TYPE
```

UI element 410 allows the user to submit the application for upload. In order to allow the document to be submitted, the UI 400 may require the user to select the document type. If any required selections were not made, an error message may be provided to the user that gives instructions as to how to remedy the error.

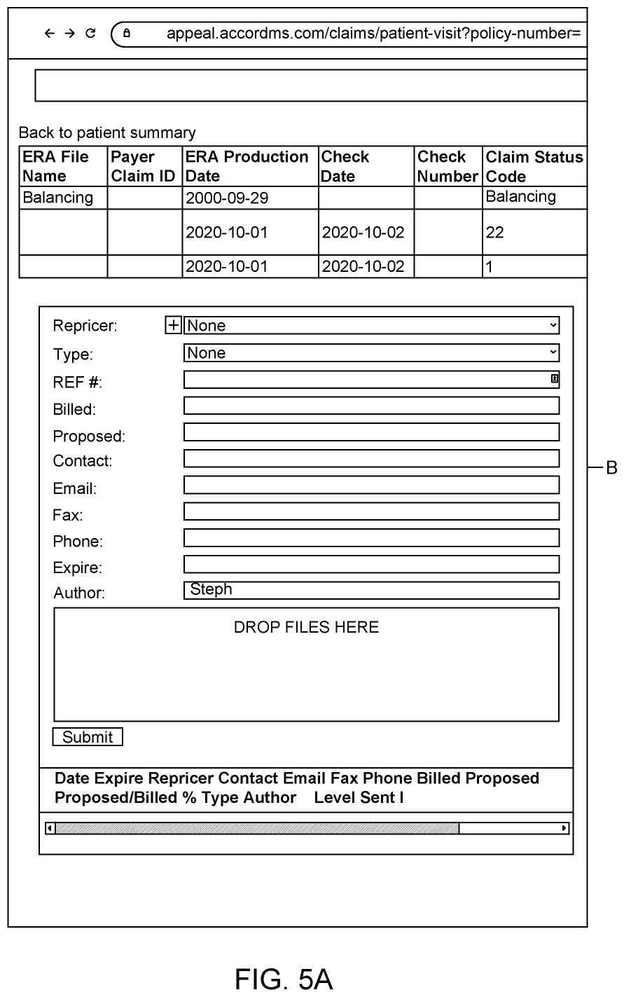

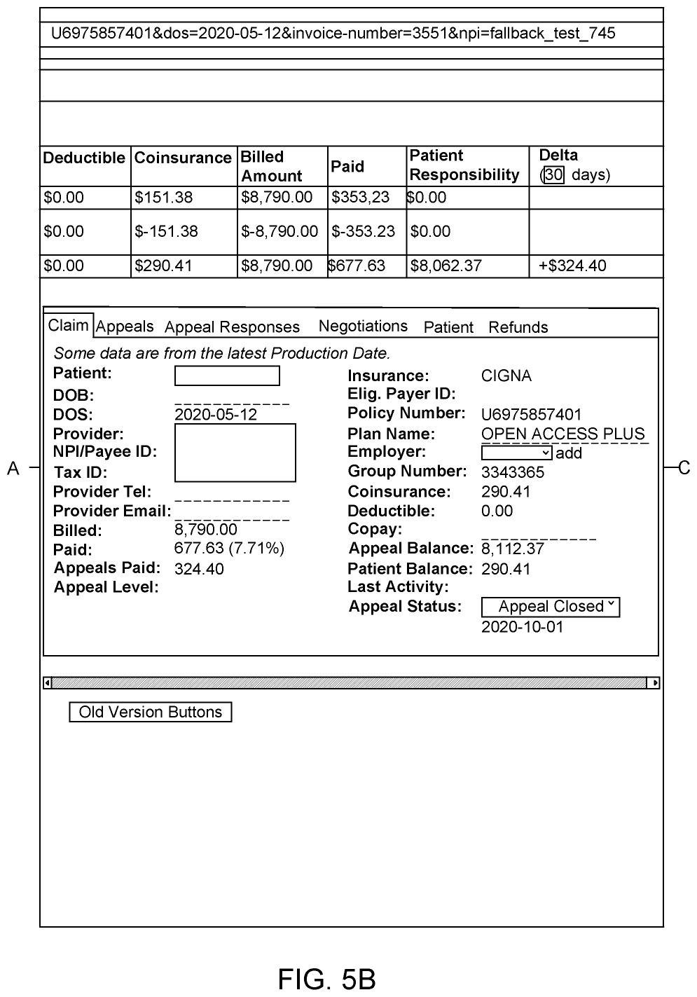

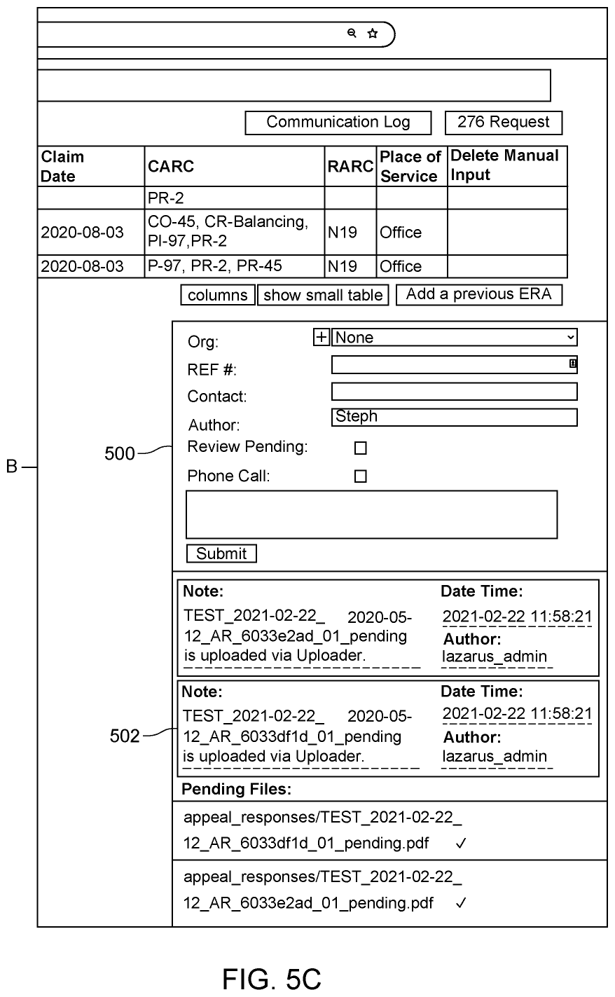

FIGS. 5A-8B are diagrams illustrating a sequence of exemplary user interfaces for managing healthcare workflows according to an embodiment of the subject matter described herein.

FIGS. 5A, 5B, and 5C are diagrams illustrating portions of an exemplary user interface, including a practice summary table UI, for managing healthcare workflows according to an embodiment of the subject matter described herein. In FIG. 5C, in a first portion of the UI is a box 500 where new phone calls and follow up activity can be recorded. Additionally, future phone calls and future follow up activity can be scheduled and will appear in a new location called “Task Manager: Phone Calls Due, Follow Up Due”. This activity may continue to be recorded in box 500 as well.

Various notes and other information may be displayed in UI portion 502 located below box 500.

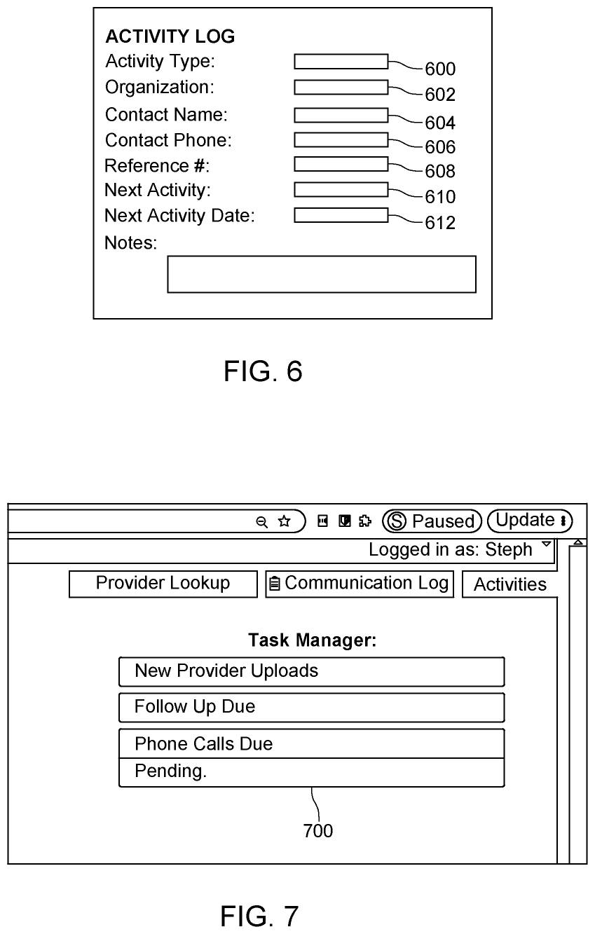

FIG. 6 is an illustration of an exemplary activity log 600. The activity log 600 may include fields including: Activity type, organization, contact name, contact phone, reference number, next activity, next activity date, and notes. Drop down options for activity type 602 may include Follow Up and Phone Call. Organization field 604, Contact Name field 606, Contact Phone field 608, and Reference number field 610 may each allow for user text entry. Next Activity field 612 may include drop down options Follow Up and Phone Call. A calendar may also be displayed for allowing the user to set a due date for the next activity. On the due date, the activity will then appear on the Task Manager under Follow Up Due or Phone Calls Due.

FIG. 7 illustrates an exemplary Task Manager interface 700. When scheduled, “Follow Up” and “Phone Calls” should appear here.

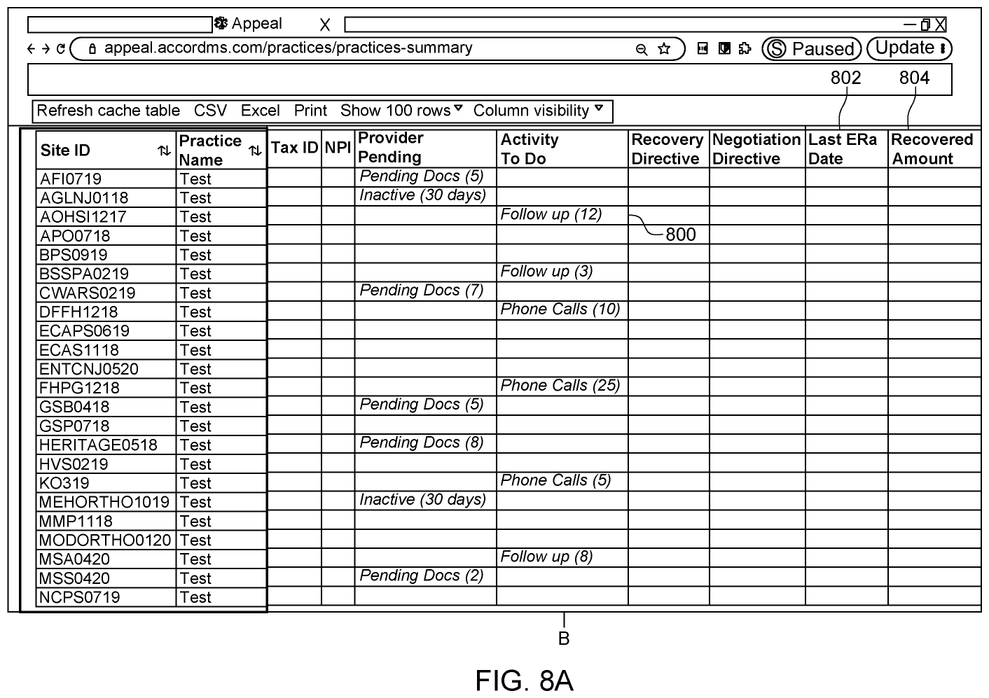

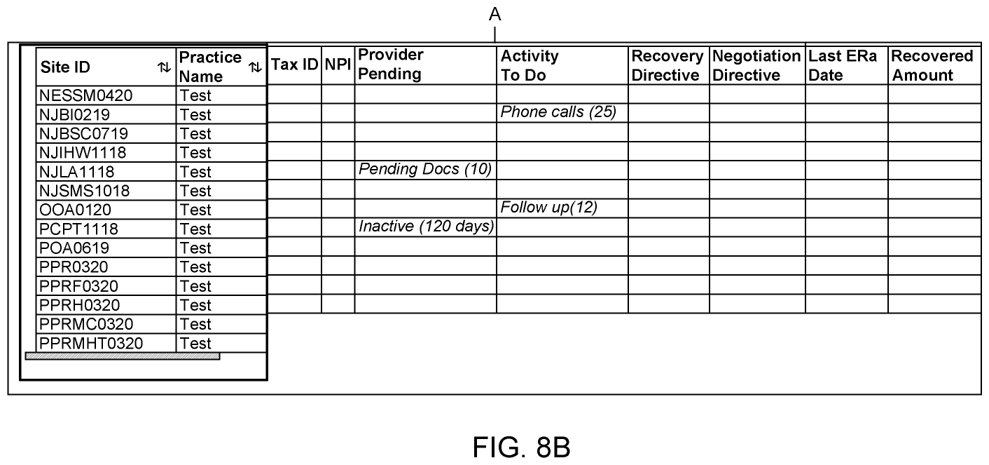

FIGS. 8A-8B illustrate an exemplary practice summary table 800. Practice summary table 800 may display data in one or more rows and columns. Here, columns include Site ID, Practice Name, Tax ID, NPI, Provider Pending, Activity To Do, Recovery Directive, Negotiation Directive, Last ERA date, Recovered Amount. The Last ERA Date is the date that an ERA was received for the client. The Recovered Amount is the amount recovered for the client, expressed in dollars or other user-selectable currency.

Cell 802 shows a Follow Up activity alert. Follow up and Phone calls that come due may give an alert so the user can visually see on this UI 800 which clients have tasks due. In another embodiment, Follow Up and Phone Calls may be displayed in separate columns.

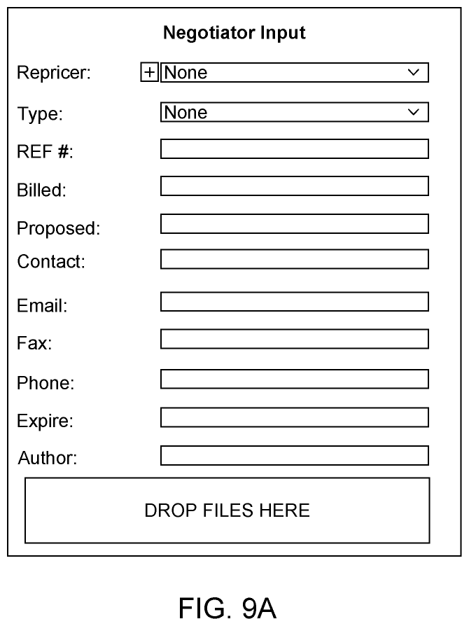

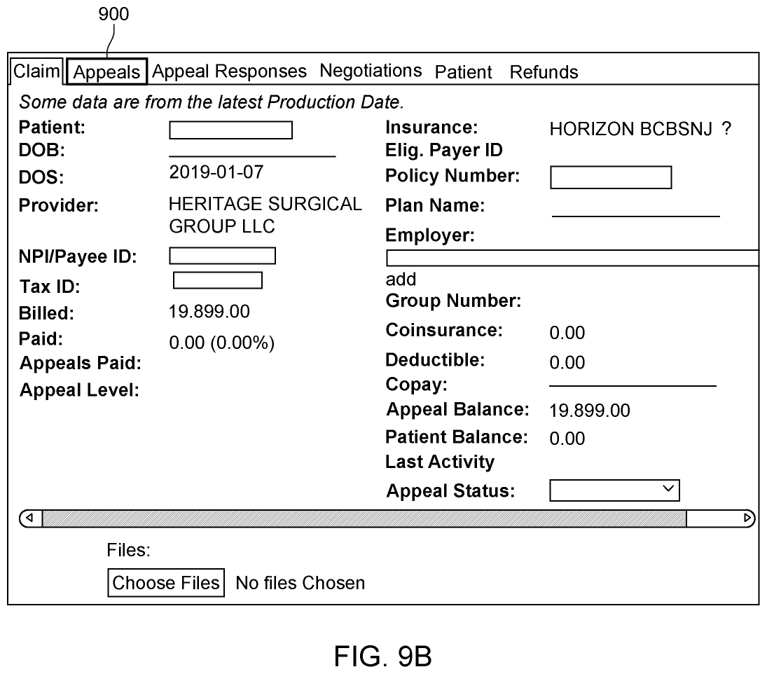

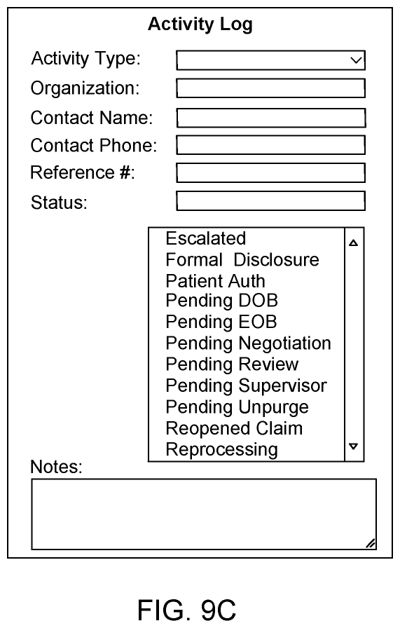

FIGS. 9A-13B are diagrams illustrating a sequence of exemplary user interfaces for filing an appeal as part of managing healthcare workflows according to an embodiment of the subject matter described herein.

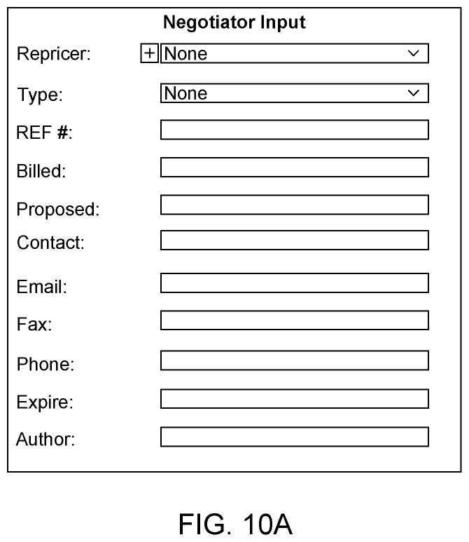

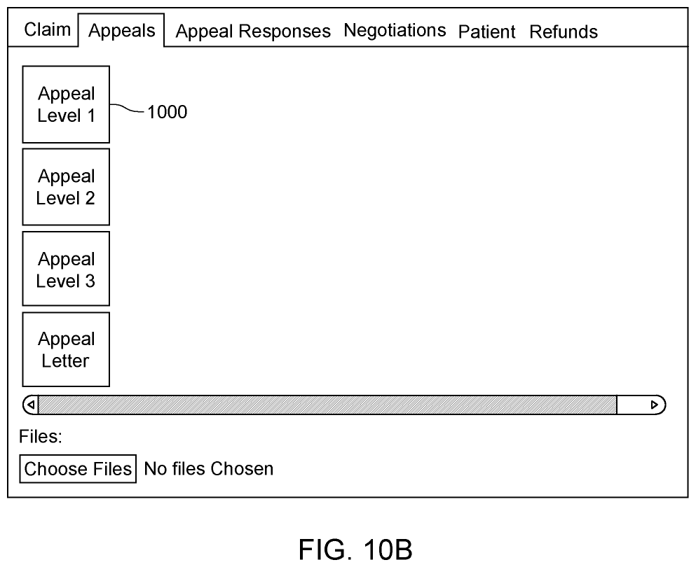

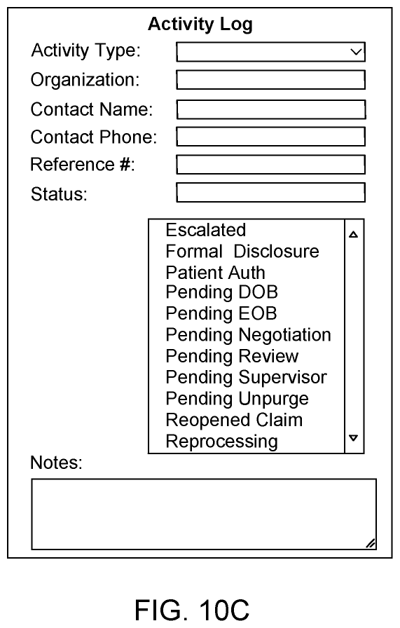

To begin an appeal, the user may begin by identifying the claim. The user may isolate claims by providing a date range and specifying a client. Next, the user may choose the correct appeal letter to be sent. Under the appeals tab 900 of the UI in FIG. 9B, the correct level of appeal letter may be selected (e.g., Appeal Level 1), as shown in selection 1000 in FIG. 10B.

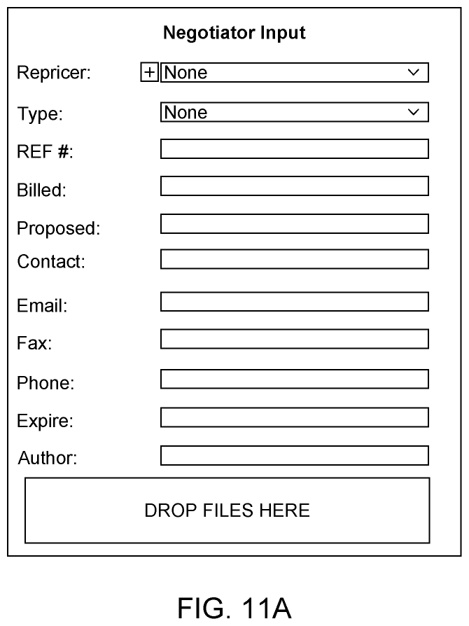

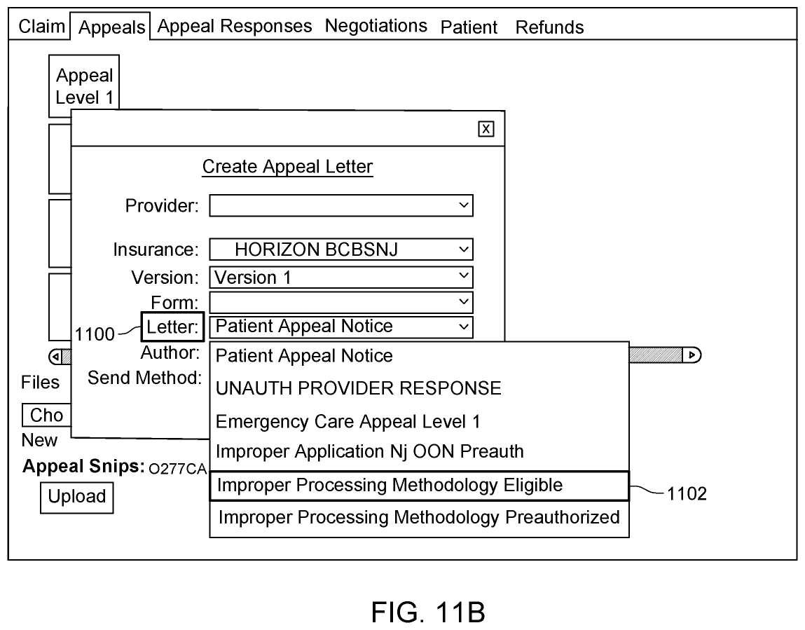

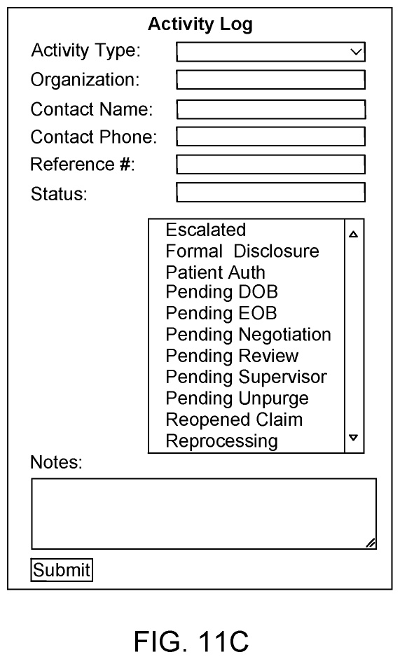

Next, as shown in FIG. 11B, the user may review and verify that information populated by the system is accurate. For example, the user may ensure that the provider, insurance, form, letter 1100, and author are all entered appropriately. Typically, the first letter 1100 to be sent out will be Improper Processing Methodology Eligible and shown in selection 1102.

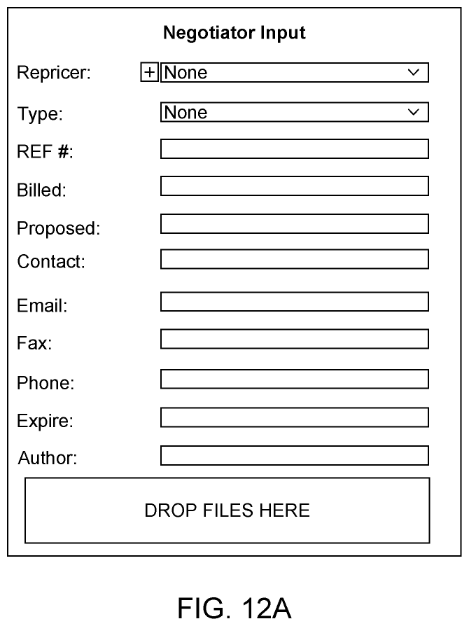

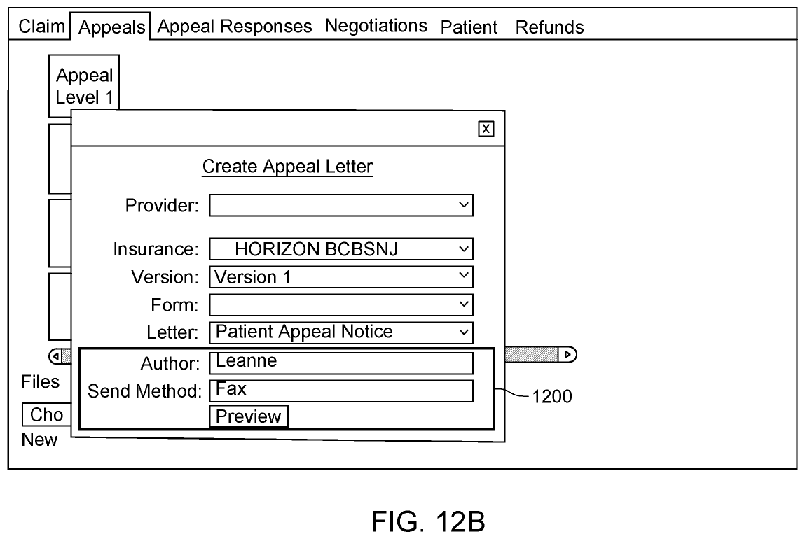

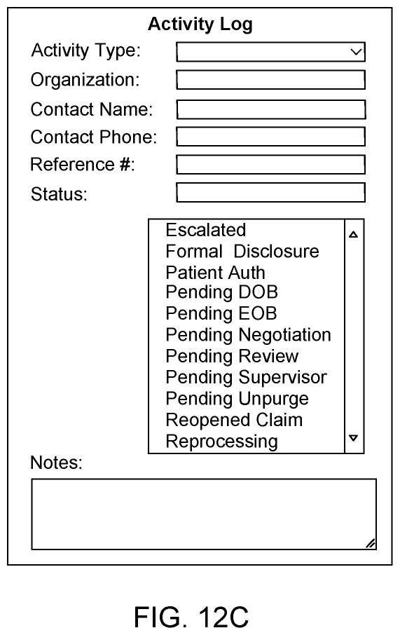

Next, as shown in FIG. 12B, the user may determine which method will be used for sending the appeal letter using UI element 1200. The most common method is “fax”. Using this method will automatically send the fax to the carrier. Another send method used is “mail”. This method is used, for example, if the carrier's fax number is not available and/or the fax delivery option may be down.

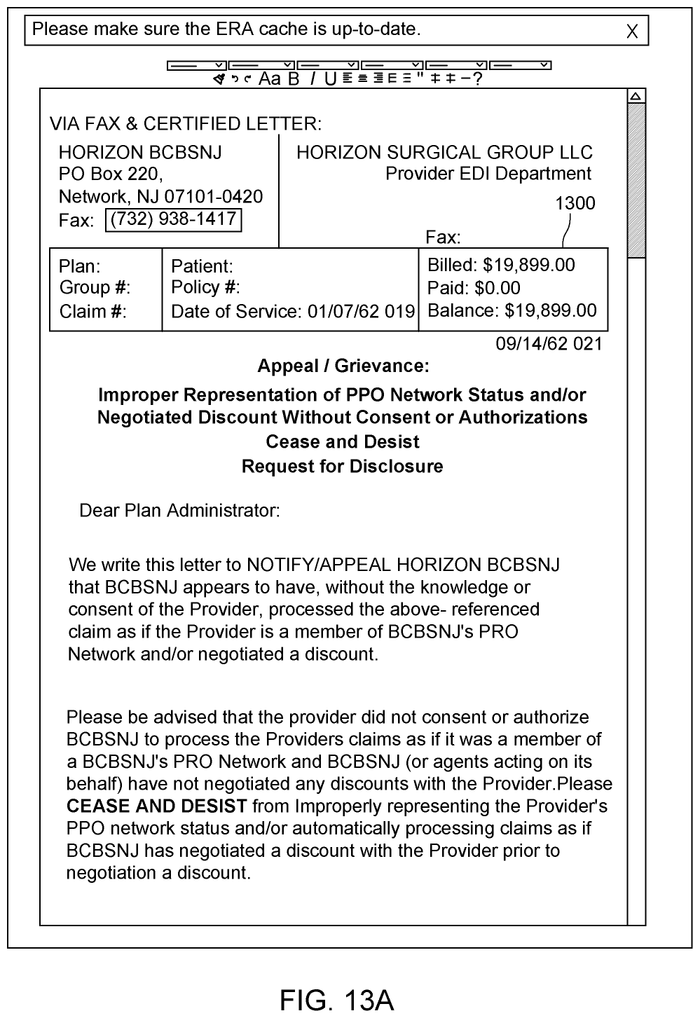

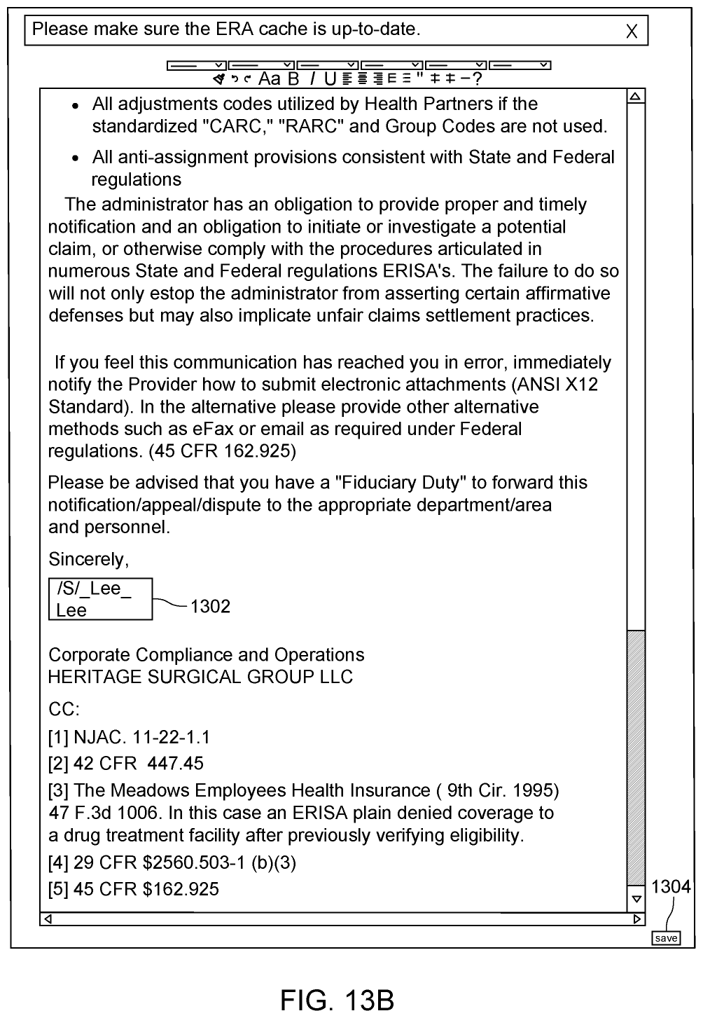

Next, in FIG. 13A, the user may review the appeal letter to certify carrier information is available (i.e., address and fax). The user may review the appeal letter to verify that all the information shown in box 1300 is correct. This may include ensuring that the provider information includes a return address and includes the provider's office address.

Next, in FIG. 13B, the user may send the appeal letter. After the letter is reviewed, the user may affix an e-signature in signature box 1302. Then clicking “save” 1304 on the bottom right-hand corner will send the appeal to the carrier.

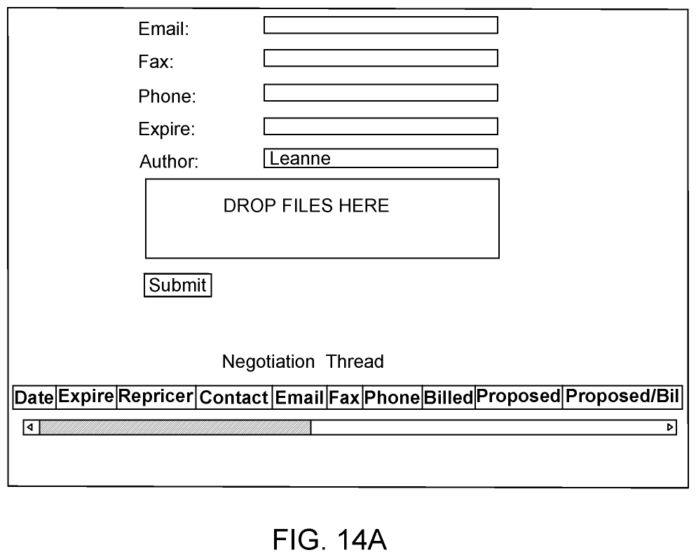

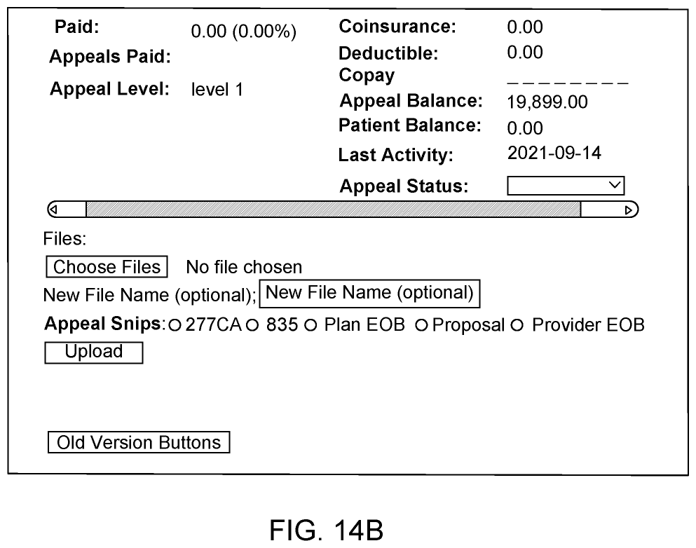

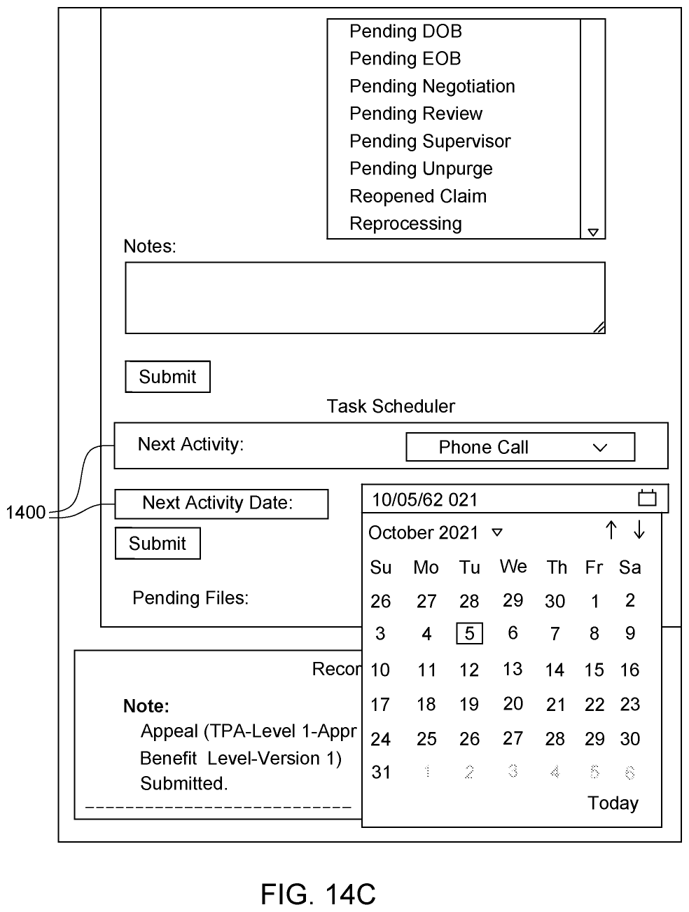

Finally, in FIGS. 14A-14C, the user may schedule a phone call. For example, using the task scheduler in the database, the user may schedule a phone call for two weeks after appeal letter is sent by selecting Next Activity: Phone Call and Next Activity Date: [two weeks in the future] in task scheduler box 1400.

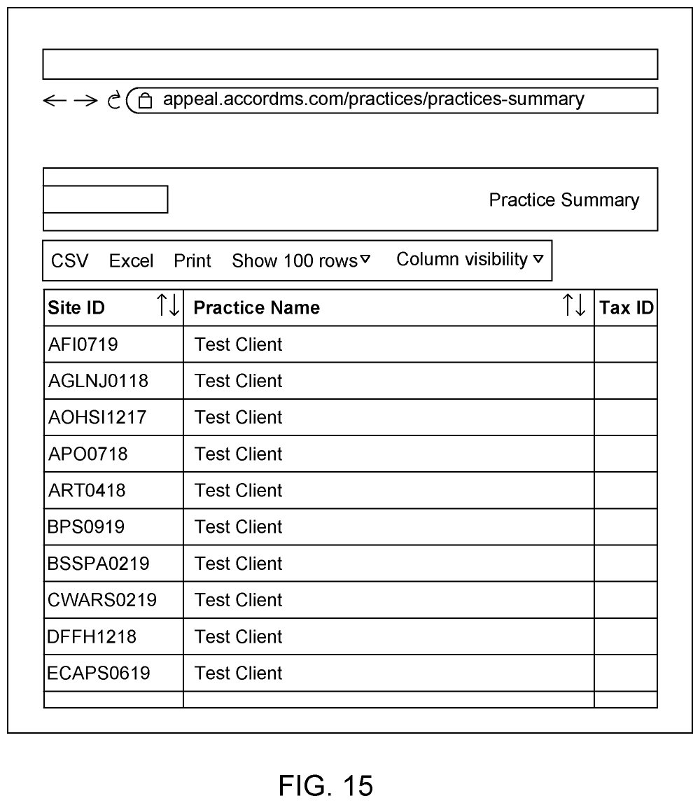

FIGS. 15-30B are diagrams illustrating a sequence of an exemplary user interface for rejecting a negotiation as part of managing healthcare workflows according to an embodiment of the subject matter described herein.

Rejecting a negotiation using the methods and systems described herein may begin on the practice summary page of an Appeals Database (ADB). For example, in FIG. 15, the user may select a Provider by double clicking on the name.

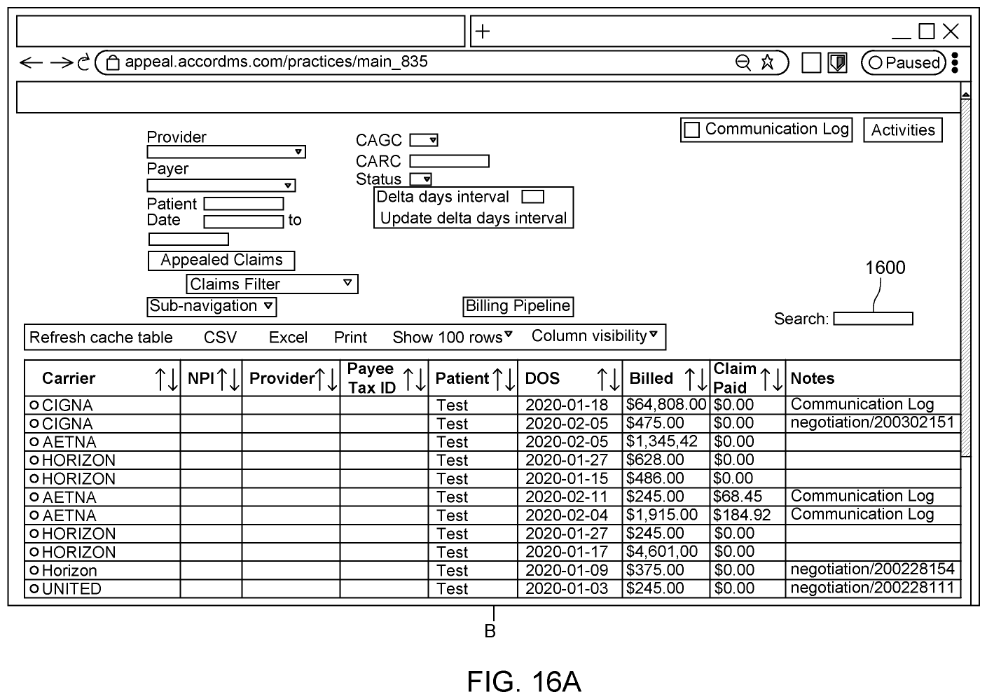

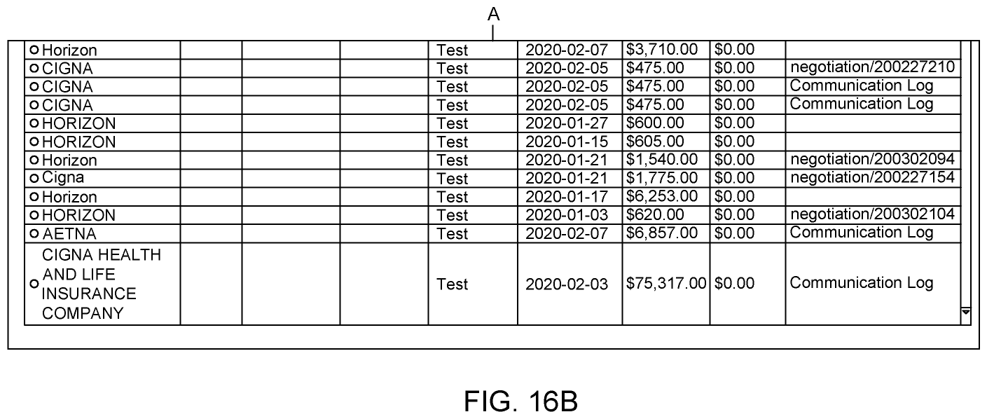

In FIG. 16A, when presented with the provider database, the user may enter the patients last name in search box 1600 to search for a claim.

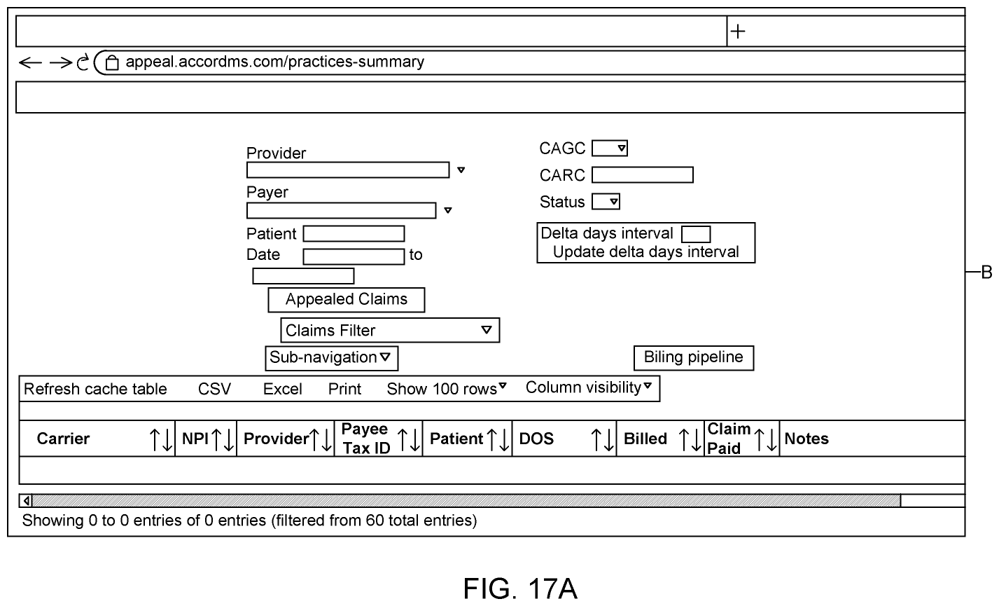

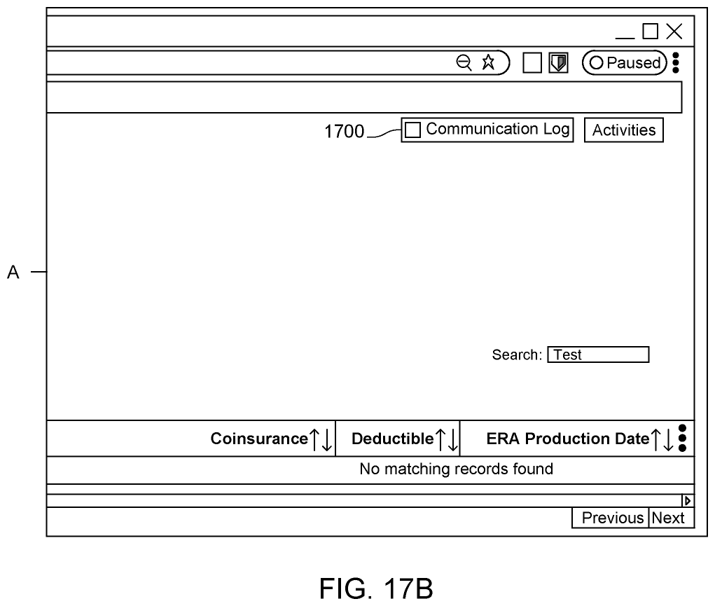

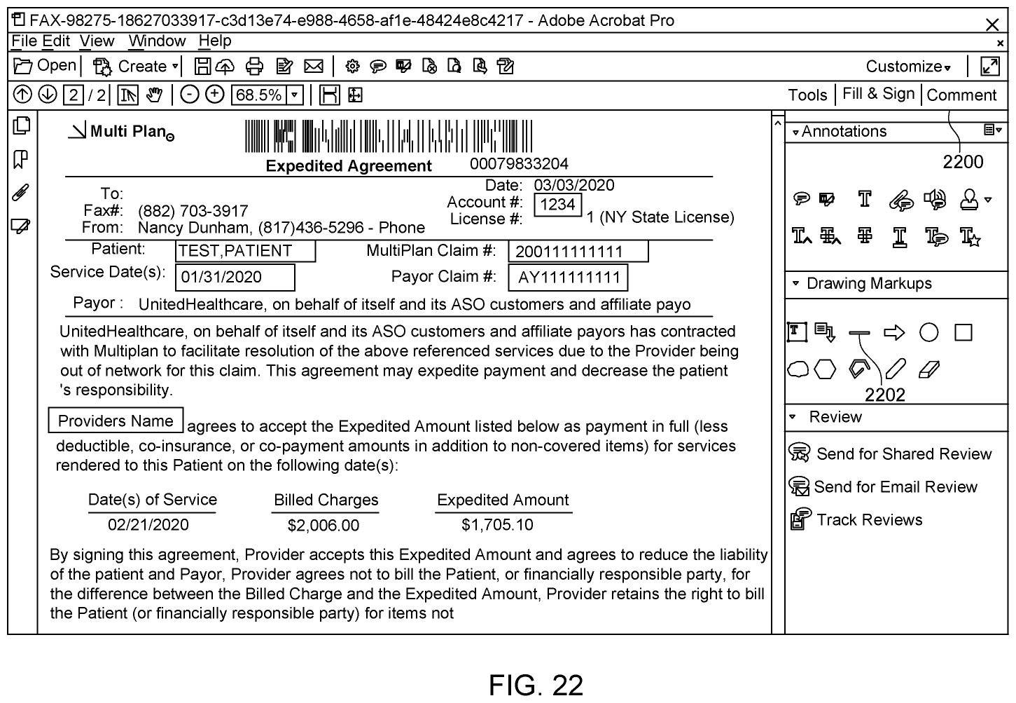

If the claim being searched for is present in the list displayed as a result of the search, the user may skip to verifying that the claim is correct (shown in FIG. 22 discussed below). If, however, the claim is not already in the database, the search will return with “No matching records found”. In this case, the user will need to manually enter the information. To perform a manual entry, the user may click on the blue box titled “Communication Log” 1700 in FIG. 17B.

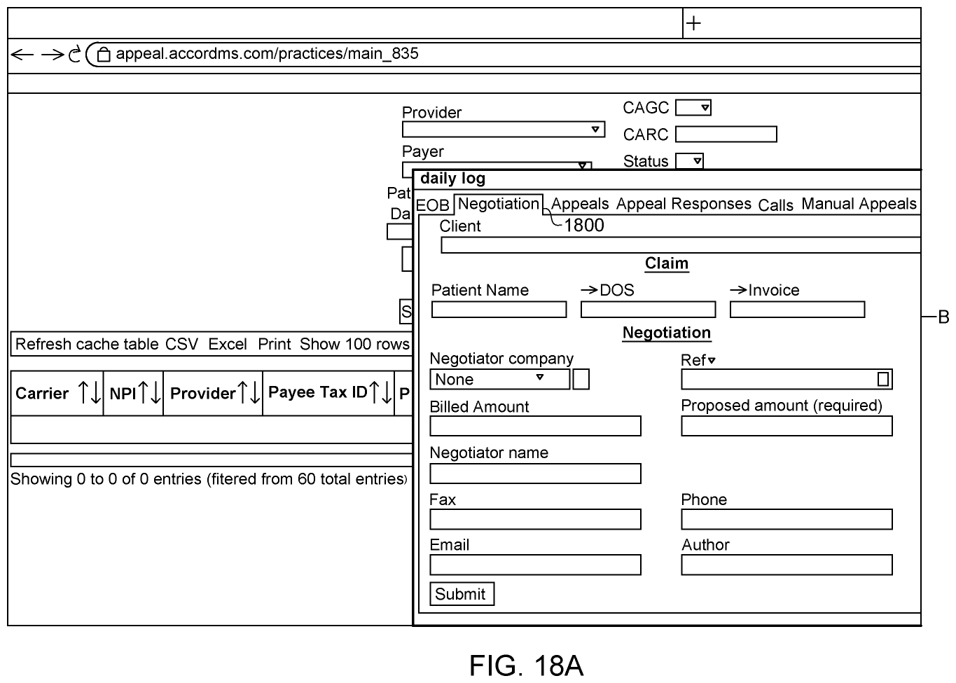

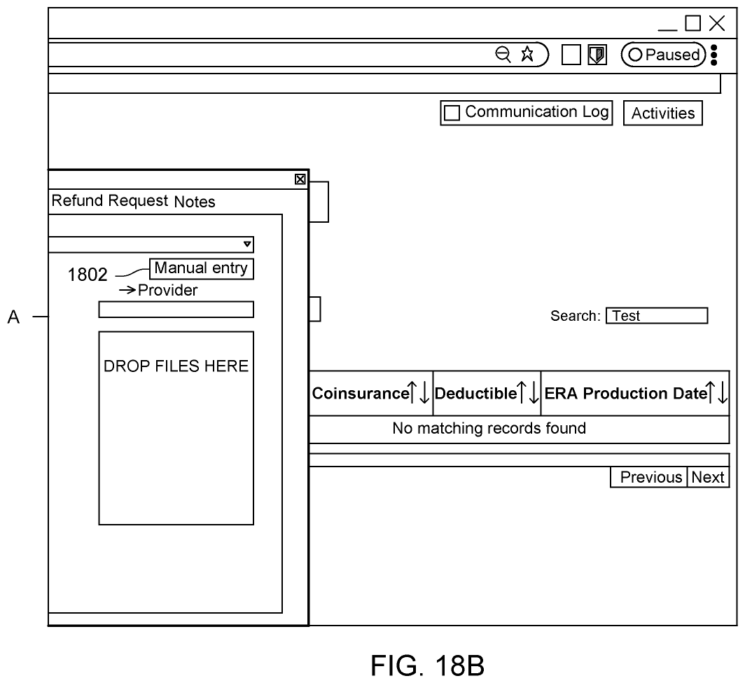

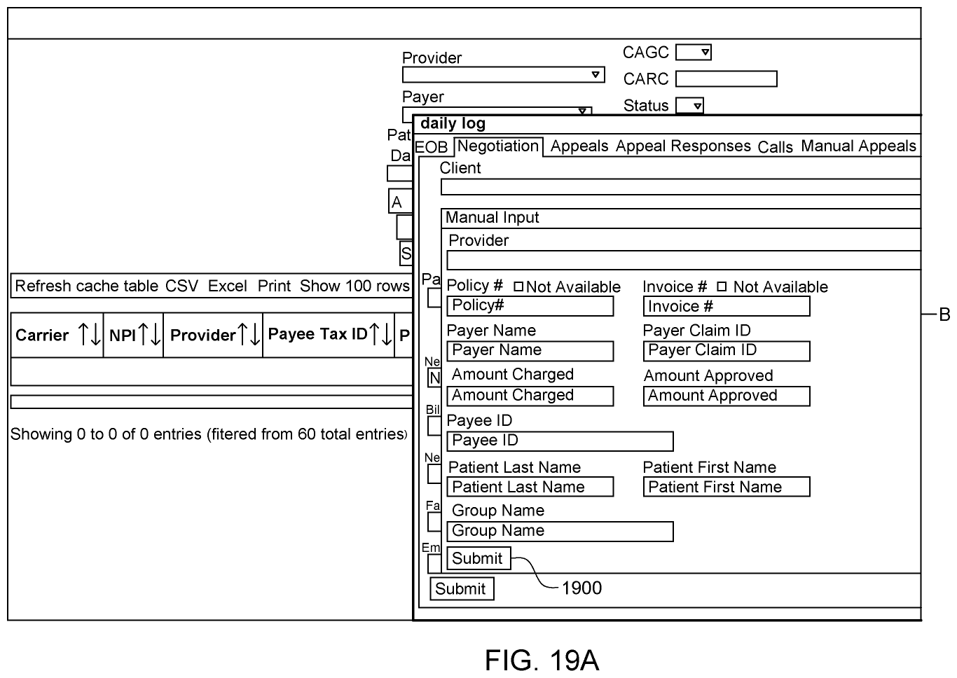

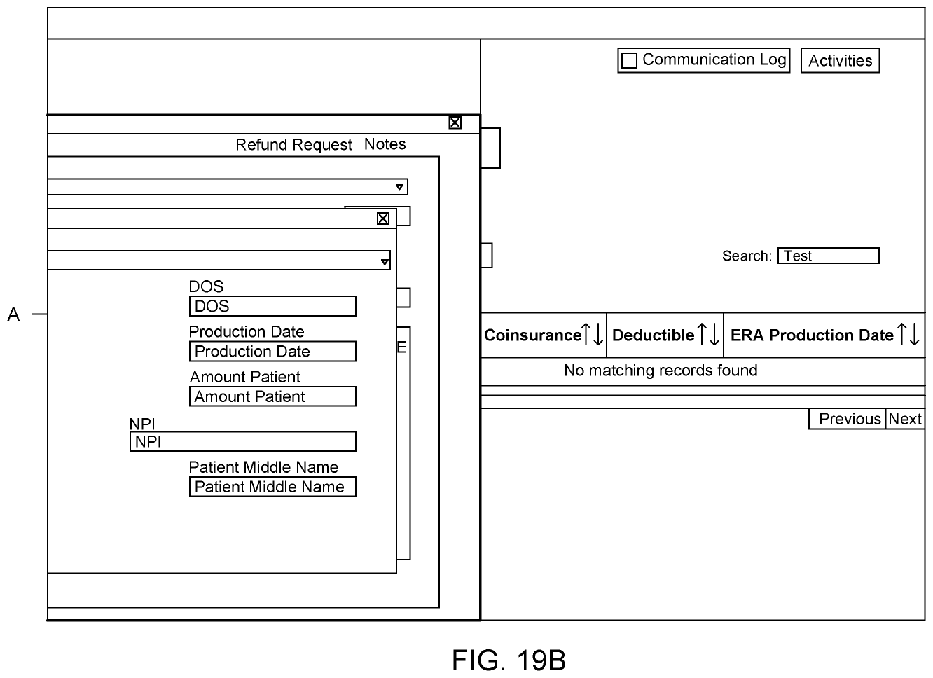

In FIGS. 18A-18B, a window titled “daily log” will open, and the user selects the tab “Negotiation” 1800, on the top row of menu options. Then the user clicks the blue button “manual entry” 1802. From this, another window titled “Manual Input” will open, as shown in FIGS. 19A-19B.

In FIGS. 19A-19B, in the Provider dropdown box, the user selects the Practice Name and does not select a doctor's name. This is because the user may not know which doctor provided the service. Various fields are presented to the user for manually entering the information. These fields, as illustrated in FIGS. 19A-19B, may include the following:

The Policy #field may be used for entering the Member ID, which in almost all cases the user will not have. If the user does not know the Member ID, the user clicks the box “Not Available”, which will generate a generic policy number.

The Invoice #field may be used for entering the account number that the provider generated when they created the claim. For example, MultiPlan may refer to this as “Account #:”). If the account number is not available from another negotiator company, the user selects “Not Available”, which will generate a generic number.

The DOS field may be used to enter the Date of service in the following format: YYYY-MM-DD. For Example, 2020-03-31.

The Payer Name field may be used to enter the name of the insurance company. This field allows the user to enter a short version of the name. For example, HORIZON, UNITED, or CIGNA.

The Payer Claim ID field may be used to enter the claim number generated by the insurance company.

The Production Date field may be used to enter the date that the negotiation was faxed to the user. This field may use the date stamp on the top of the fax in YYYY-MM-DD format.

The amount charged field may be used to enter the billed amount.

The amount approved field should be “0.00”.

The amount patient field should be “0.00”.

The Payee ID field may be used to enter the providers Tax ID number, which can be found at the top of the provider page in the black header).

The NPI field may be used to enter the provider's GROUP NPI number. This can be found at the top of the provider page in the black header.

The Patient Last Name field may be used to enter the patient's last name as a string of alphanumeric characters.

The Patient First Name field may be used to enter the patient's first name as a string of alphanumeric characters.

The Patient Middle Name field may be used to enter the patient's middle name as a string of alphanumeric characters.

The Group Name field may be used to enter, if it is available, the name of the employer which the patient is covered by.

Finally, the user clicks the blue “Submit” button 1900 to complete the rejection of the negotiation.

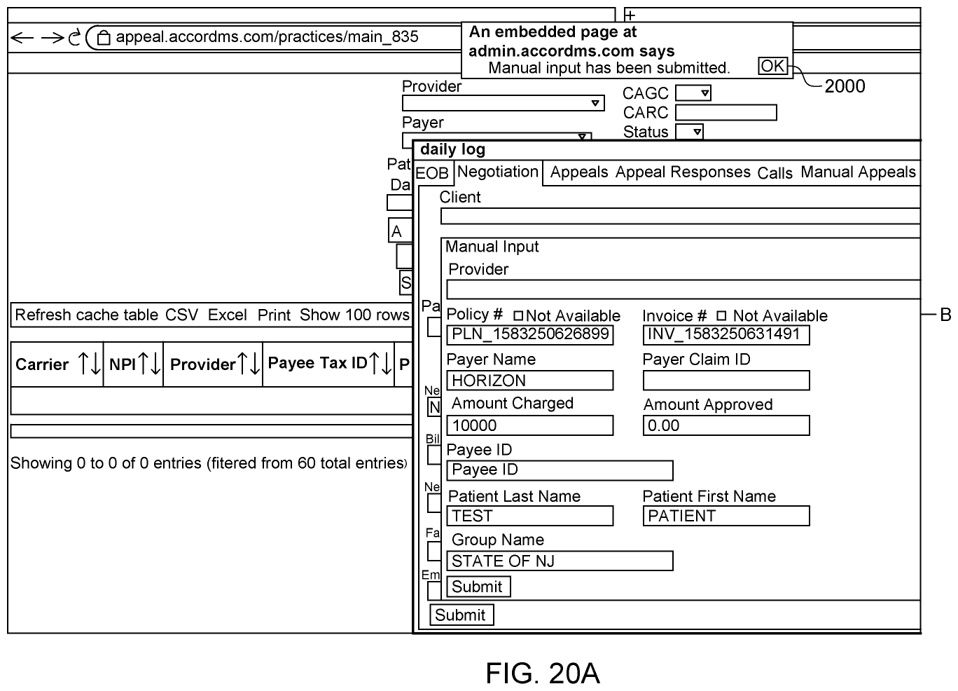

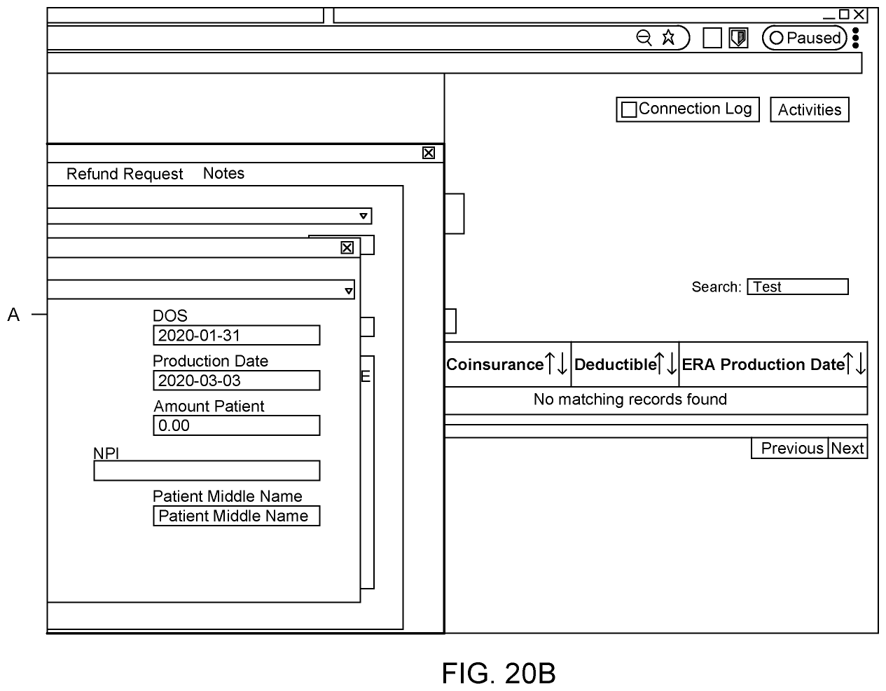

Next, in FIGS. 20A-20B, an embedded page at the exemplary URL admin.accordms.com may display a popup “Manual input has been submitted.” The user then can click the blue “OK” button 2000. The user can then close the “daily log” window by clicking on the “x” in the upper right corner of window.

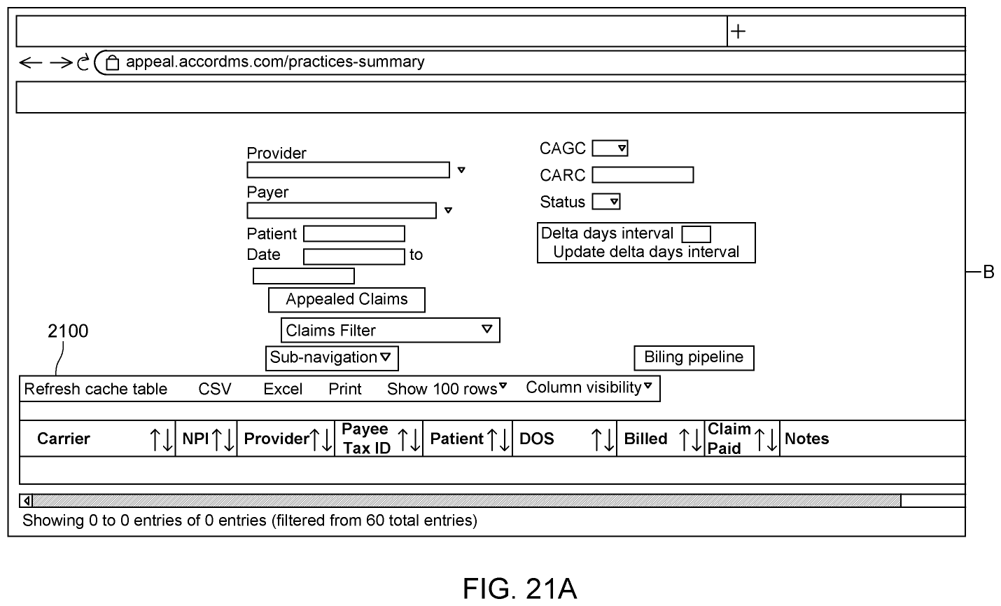

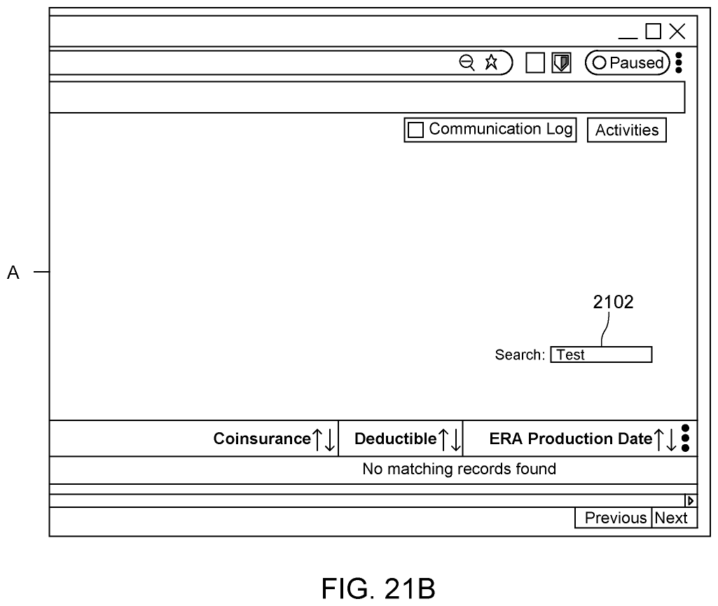

In FIGS. 21A-21B, the user can Click on the “Refresh cache table” 2100 on the left side, black menu bar. After entering the patient's last name into the “Search” field 2102 and selecting enter, the claim entered will appear in the list. Next, the user can verify that they are clicking on the correct claim by examining the Patient Name, DOS, and Billed Amount. Then the user can double click on the patient's name, which will display the Claim details screen.

In FIG. 22, the user can make edits to the faxed negotiation pdf before uploading it to the database. For example, the user can click on “Comment” 2200 on the top right side of menu bar, then click on the red line strike tool 2202 in the Drawing Markups menu and use the cursor to draw a line striking through the “Expedited” or “Proposal Amount”.

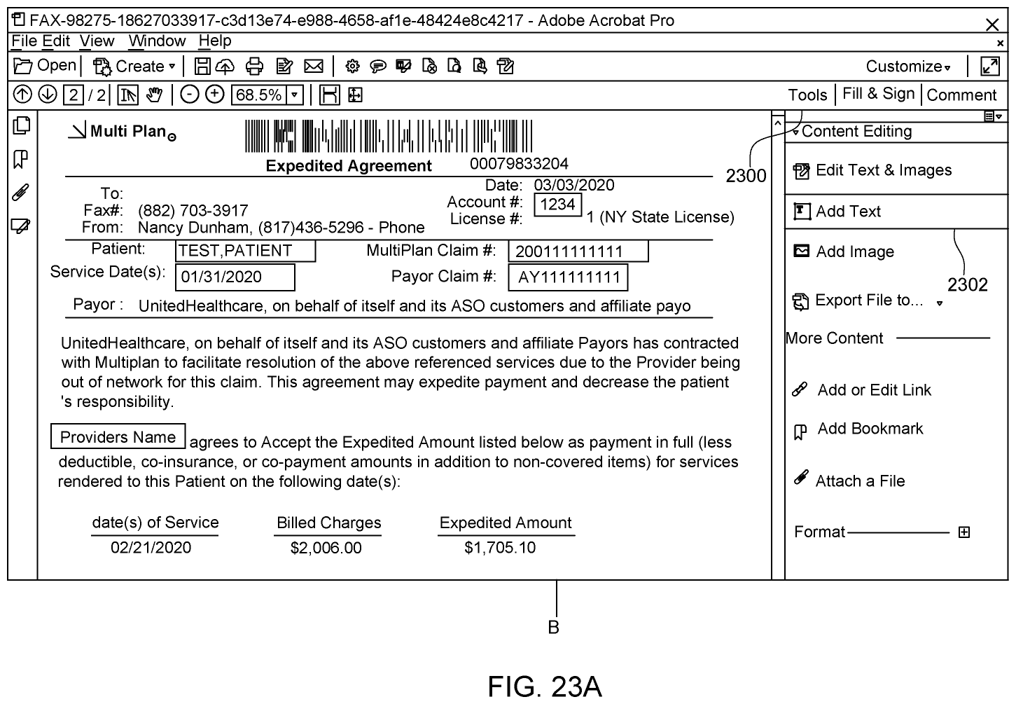

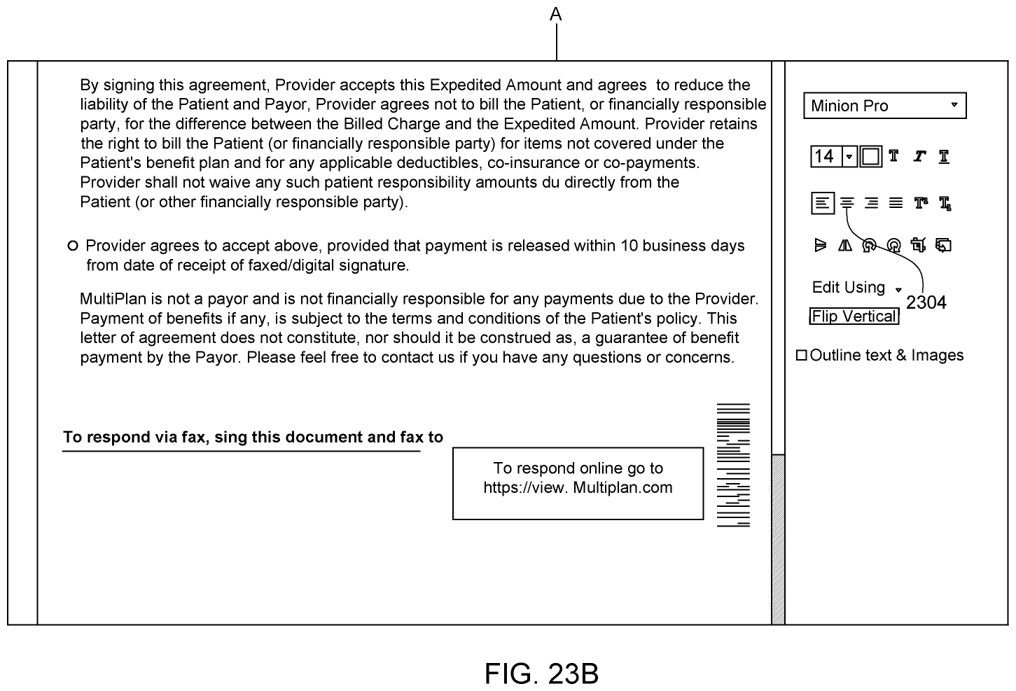

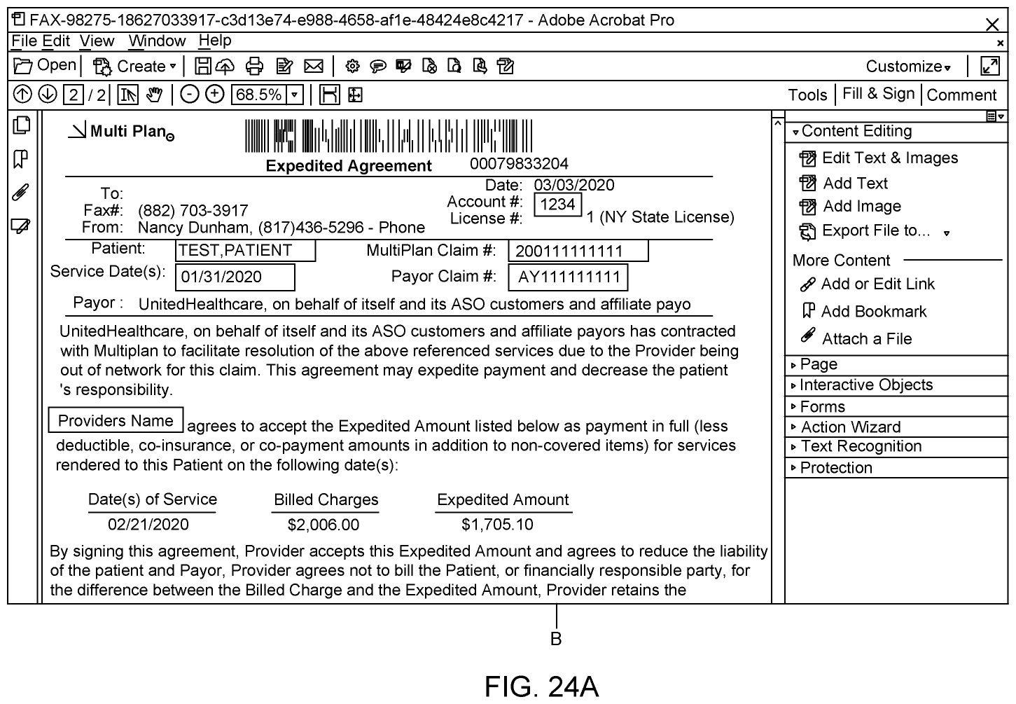

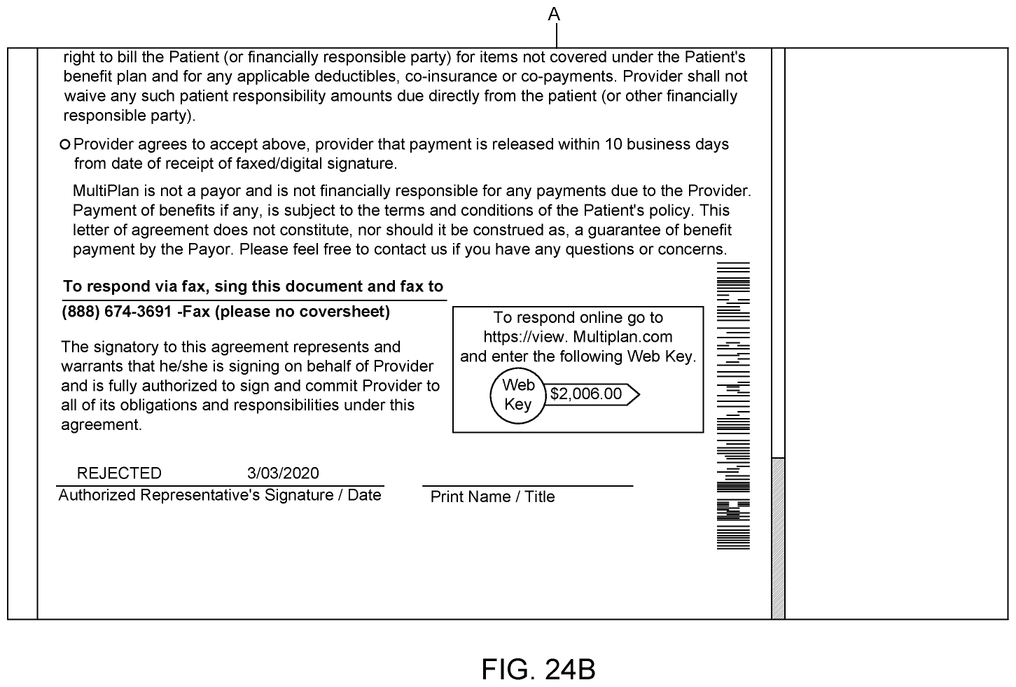

In FIGS. 23A-23B and FIGS. 24A-24B, the user can next click on “Tools” 2300 on the top right side of menu bar. Then under the Content Editing menu, the user can click on “Add Text” 2302 and change the font size to “14” and click on the black box next to it and change the color to red using formatting tools 2304. Clicking above the “Expedited Amount” will create a text box, and the user can enter the dollar amount of a counter proposal. In one embodiment, if it is the first offer, the counter proposal may automatically be determined (e.g., 85% of billed amount) and if it is a second offer, the counter proposal may automatically be determined to be a different amount (e.g., 75% of billed amount). This determination may vary based on a variety of factors.

Next, the user can click above the “Authorized Representative's Signature . . . ” and type in “REJECTED [todays date]”. When editing is complete, the user can select the “Esc” button on the keyboard twice. The user can then navigate to “File”, “Save As”, and rename the file using the following format: TESTFILE_2020-03-03_PATIENT_TEST_2020-01-31_NEG1.1R Then click save. A new file will be saved to the same fax folder and will appear at the top of the fax folder list.

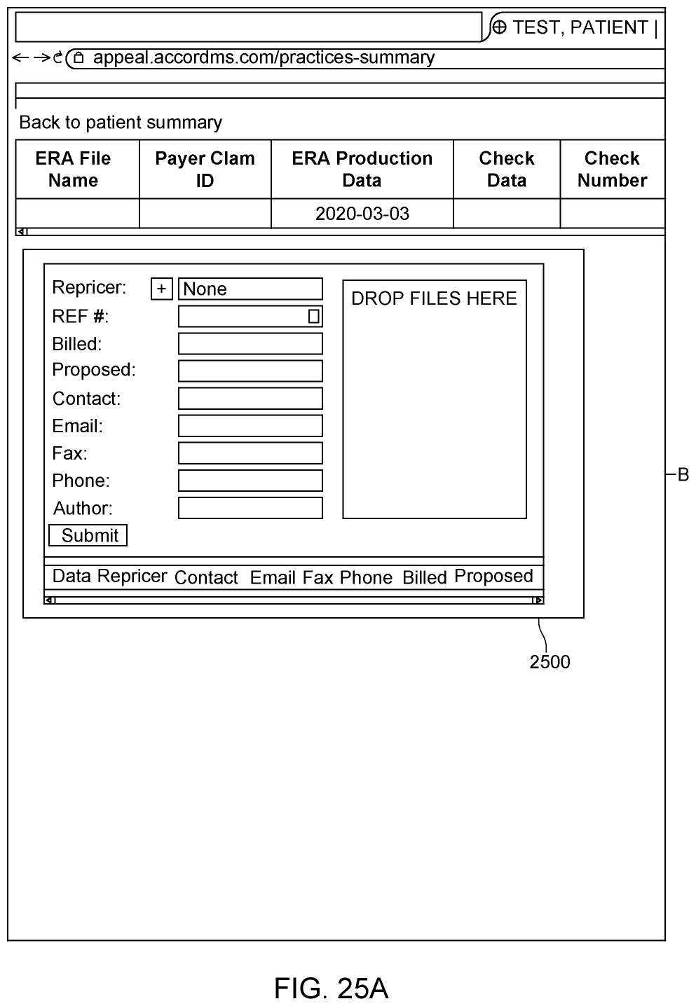

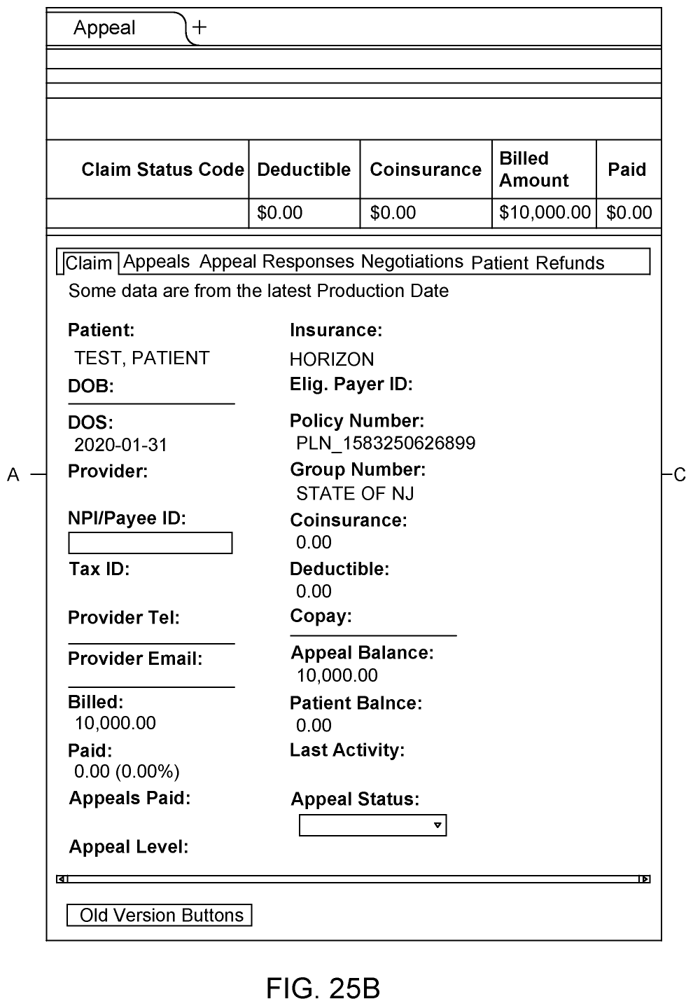

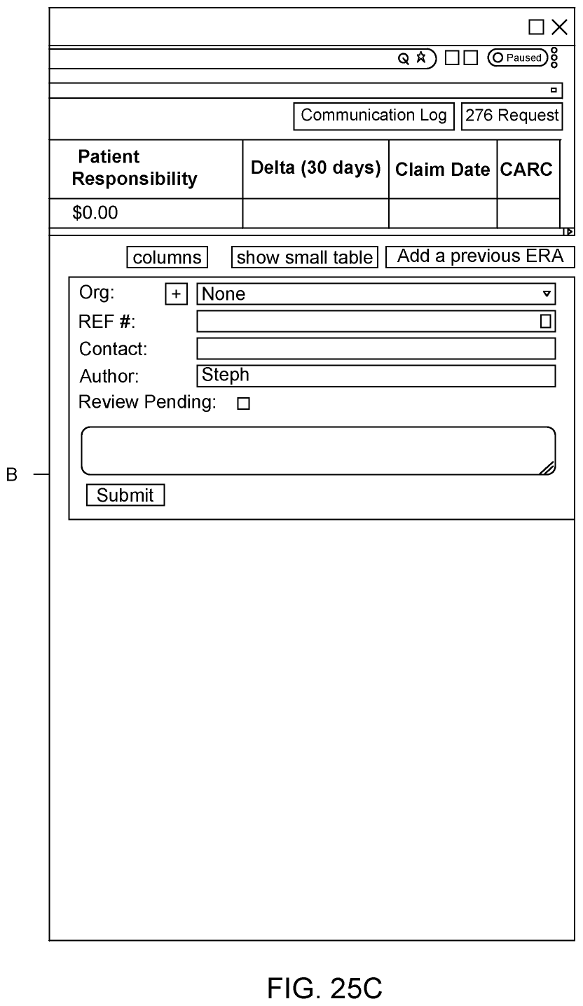

In FIGS. 25A-25C, the user can return to the provider database to enter the negotiation details and upload the file. On the left side of the screen there is a section where you will enter the Negotiation Details from the fax that was received. For example, data entry fields may include: a Repricer field for selecting the proper negotiation company from a drop down box, a reference number (REF #) field for entering the reference number generated by the negotiation company, a Billed field for entering the amount that the provider billed, a Proposed field for entering the amount that the negotiation company is offering to pay the provider, a Contact field for entering the name of the person from negotiation company that sent the fax, an Email field for entering an email address, a Fax field for enter the negotiation company fax number, a Phone field for entering the phone number of the negotiator, and an Author for entering the user's name.

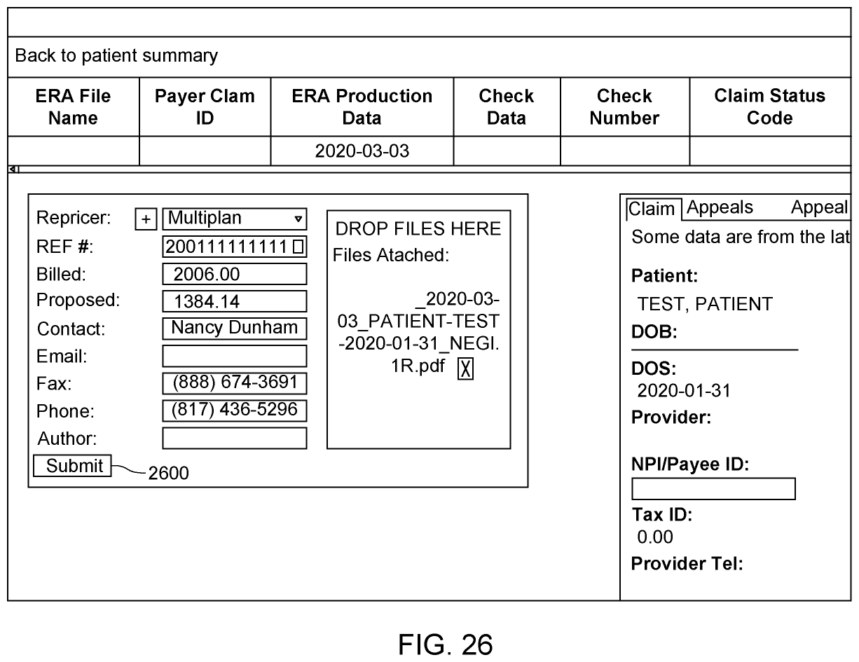

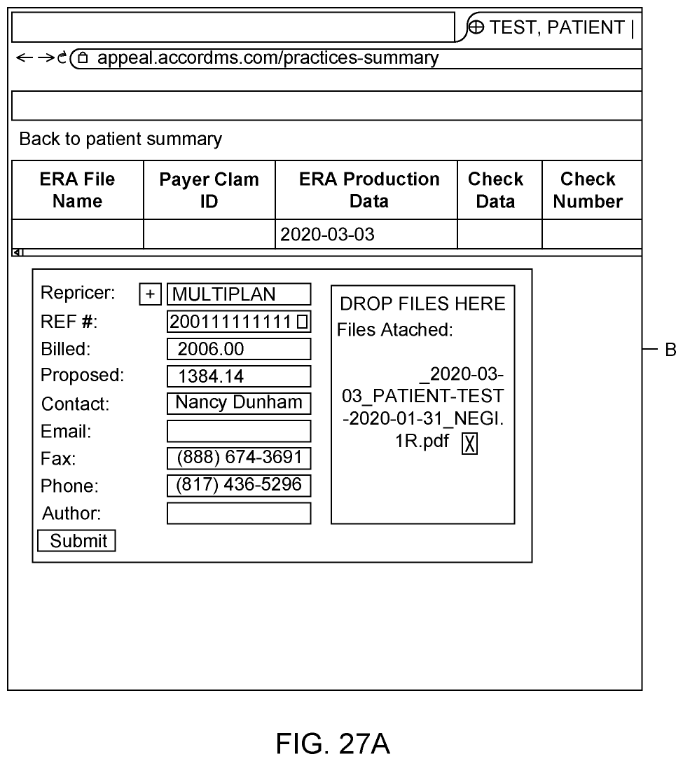

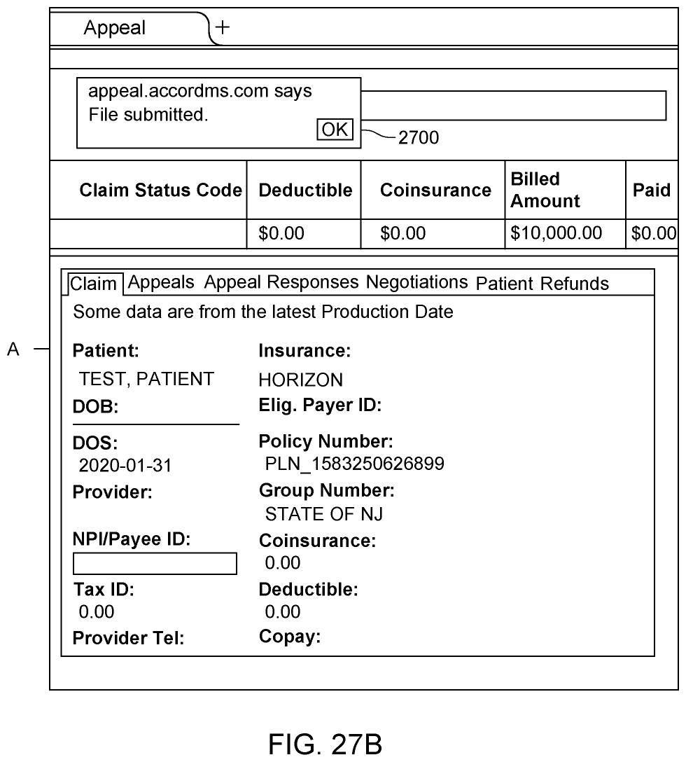

Next, the user can drag and drop the negotiation that was edited and renamed in Adobe PDF Editor by clicking on the file, dragging it to “DROP FILES HERE”, and dropping the file. If successful, the file name will be displayed in the box. If successful, the user then clicks the blue “Submit” button 2600 as shown in FIG. 26 and a dialogue box will appear at the top of page “File submitted.” The user can then click the blue “OK” button 2700 shown in FIGS. 27A-27B.

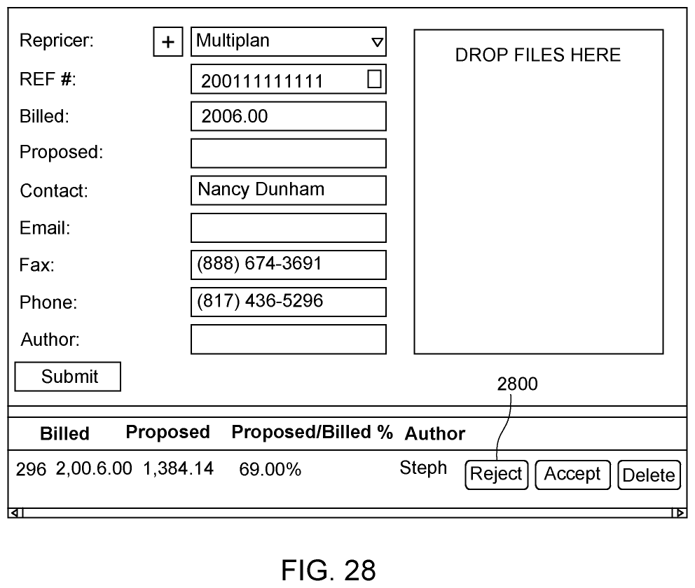

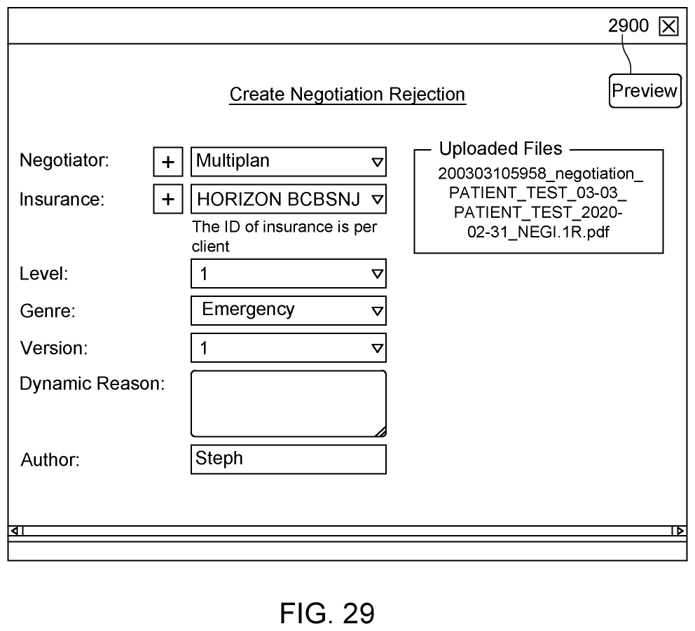

The claim data will now appear in the thread underneath and the user can scroll to the right and make sure data is correct. Next, the user can click on the blue “Reject” button 2800 shown in FIG. 28. Doing so will open a new window for creating a negotiation rejection.

In the “Create Negotiation Rejection” screen shown in FIG. 29, the user can verify that the following data fields are correct: Negotiator company, Insurance company, Level (e.g., level 1 if it is the first letter, Level 2 if it is the second letter), Genre (e.g., Emergency), Version (e.g. 1), Dynamic Reason, and Author. Once verified, the user can click the blue “Preview” button 2900 in the upper right corner.

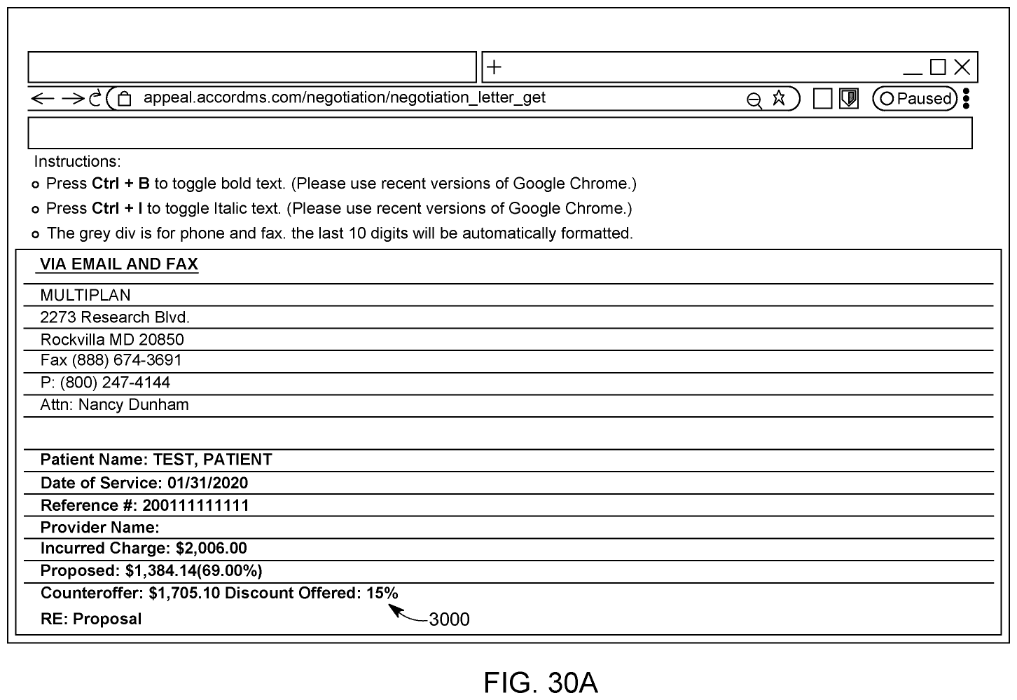

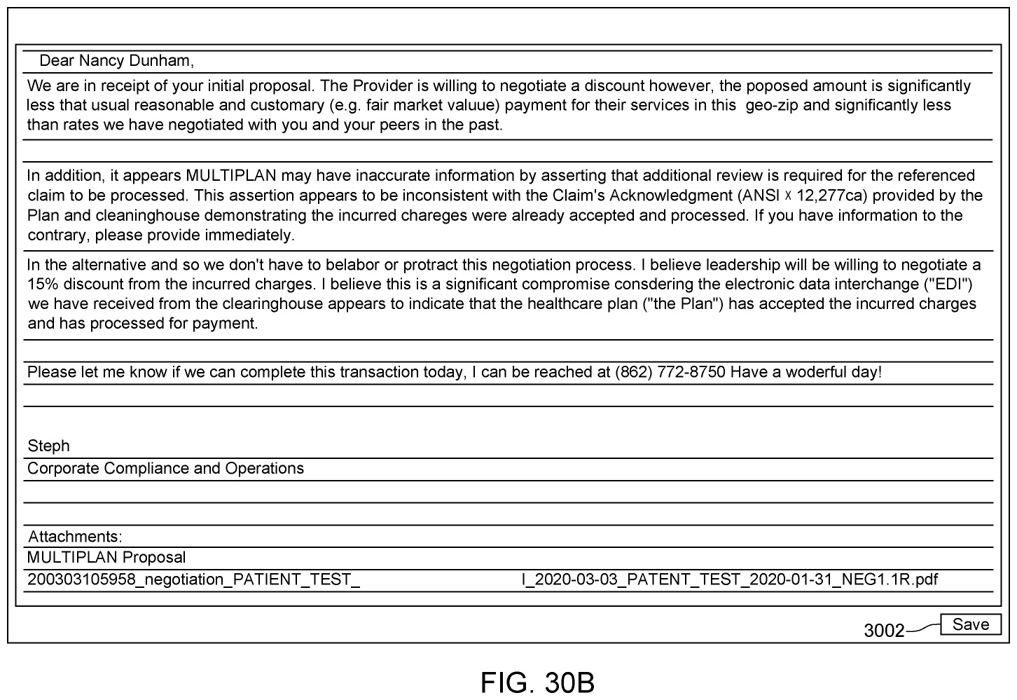

In FIGS. 30A-30B, the user can view and edit the negotiation letter here. For example, it may be important to verify that the counteroffer and discount match with the information that was edited into the pdf. Here, a period should be added to the second to last sentence 3000. Then the user can click the blue “Save” button 3002 in the lower right corner of the screen.

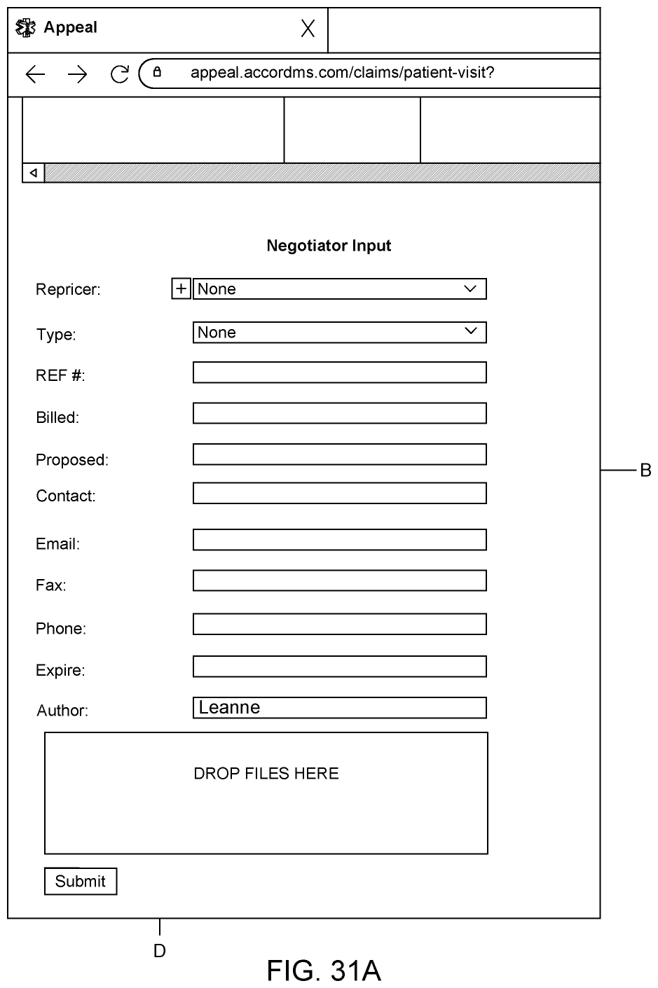

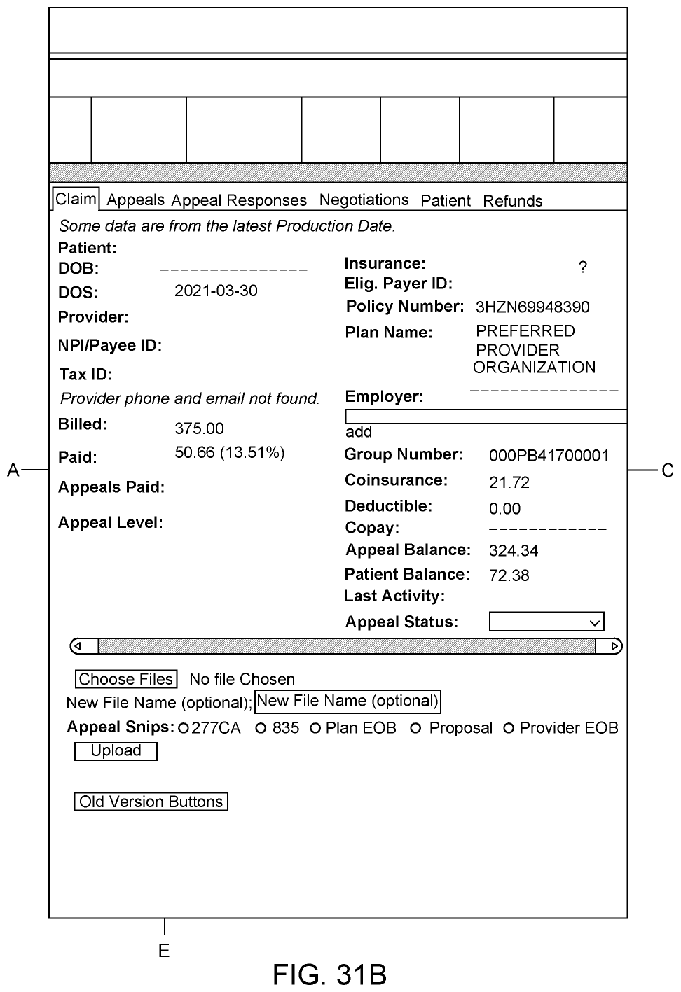

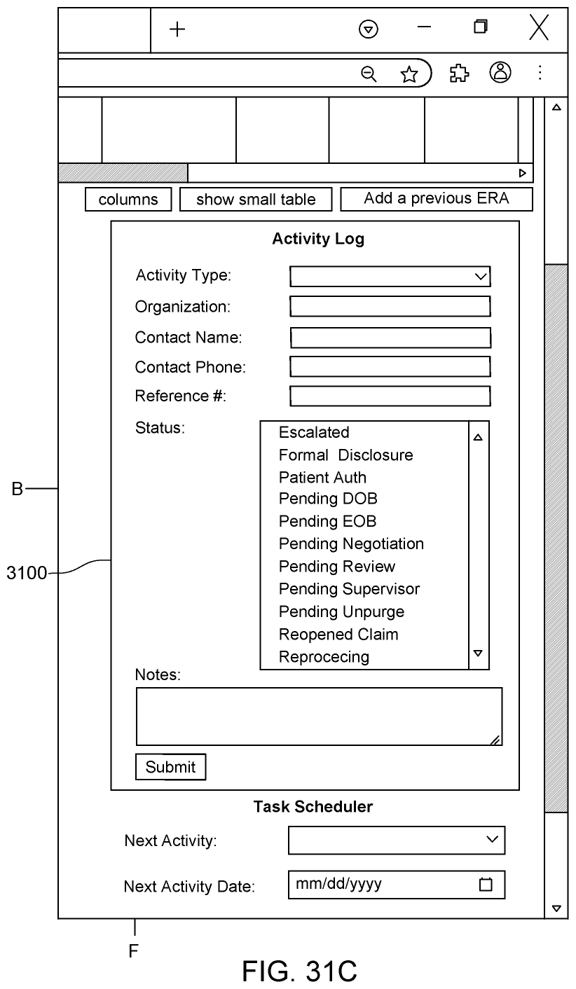

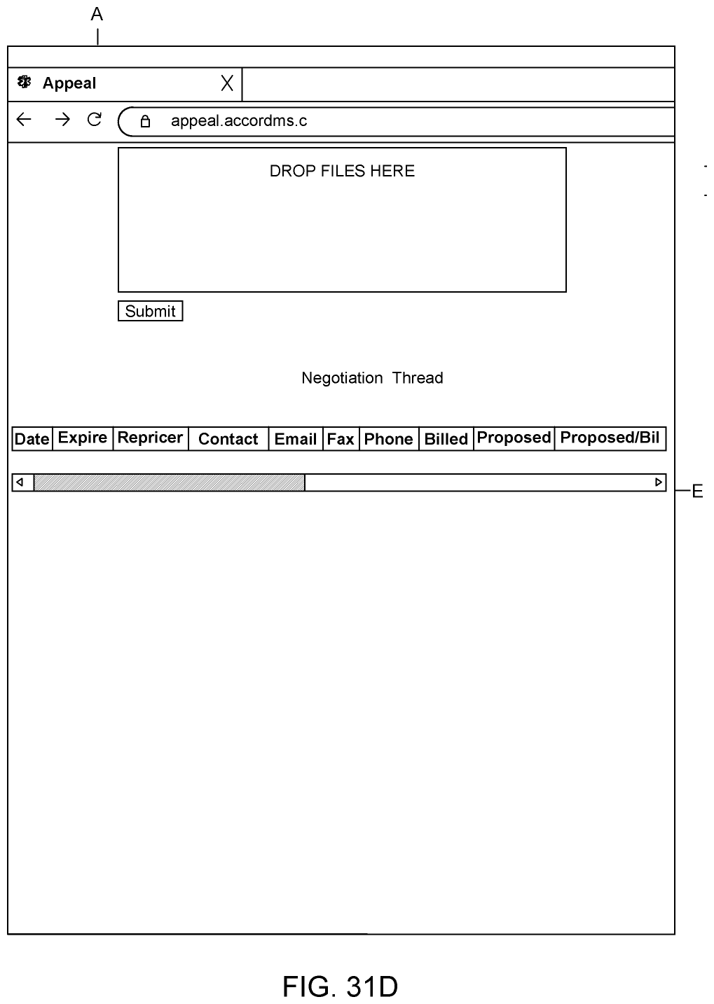

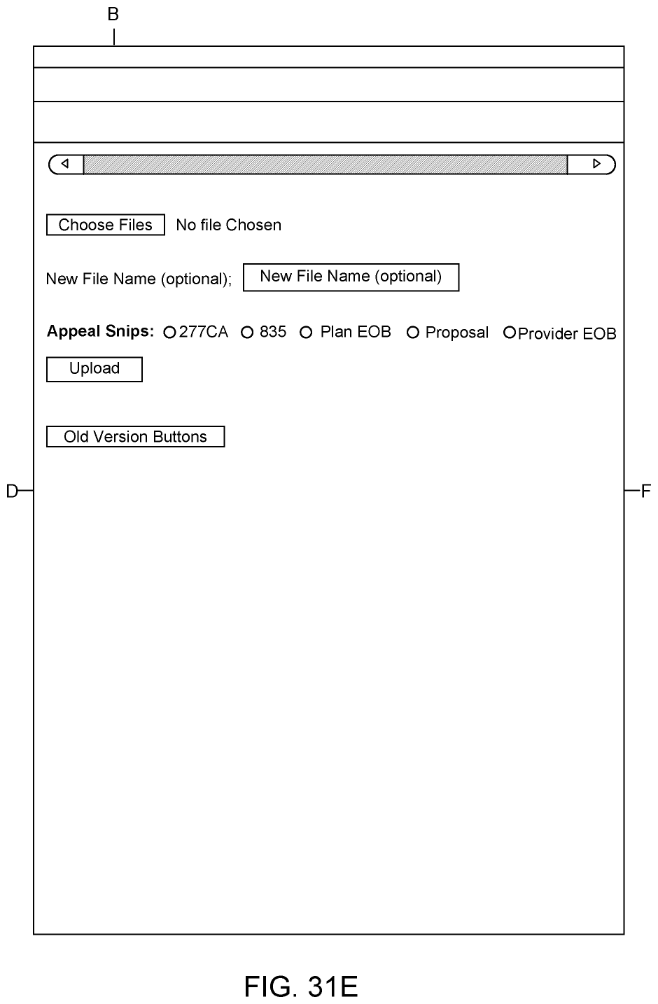

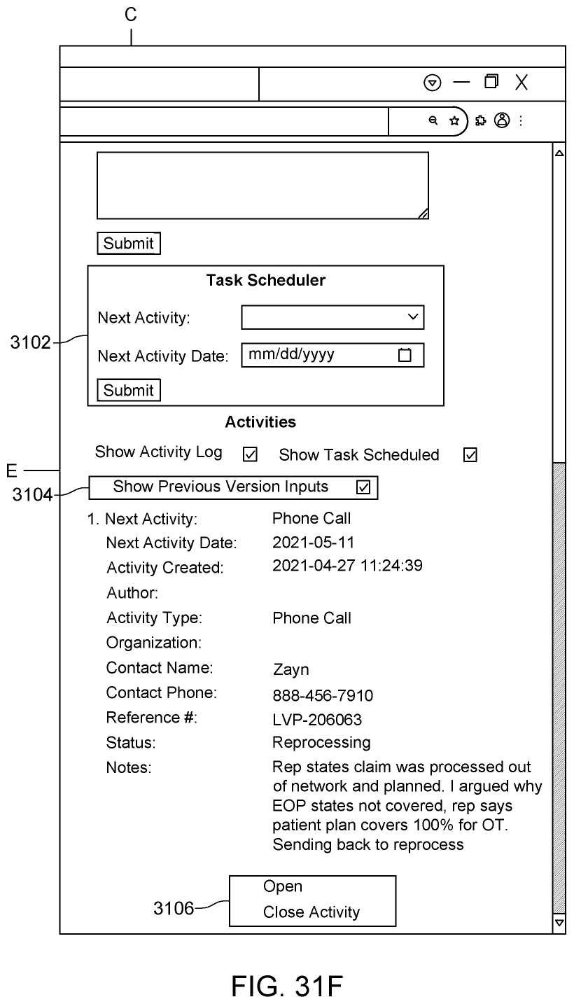

FIGS. 31A-31F illustrate an exemplary user interface for documenting phone calls as part of managing healthcare workflows according to an embodiment of the subject matter described herein.

In FIG. 31C, the UI may include activity log 3100 where the user can choose one or more statuses and fill out each line as applicable. Before moving on to the Task Scheduler, the user may click Submit here to avoid losing information entered in the Notes section.

In FIG. 31F, the UI may include Task Scheduler 3102 where the user can choose a phone call as the next activity and schedule the next activity date for two weeks in the future. Below Task Scheduler 3102, Activities 3104 allows the user to select what information is displayed. Options include show activity log, show task scheduled, show previous version inputs. The show previous version inputs selection may be used if the user cannot view previous notes. At the bottom of the UI shown in FIG. 31F, the user can either open or close the activity using interface element 3106. It may be important for the user to “close activity” on previous calls, if applicable, to prevent claims from incorrectly showing up as due.

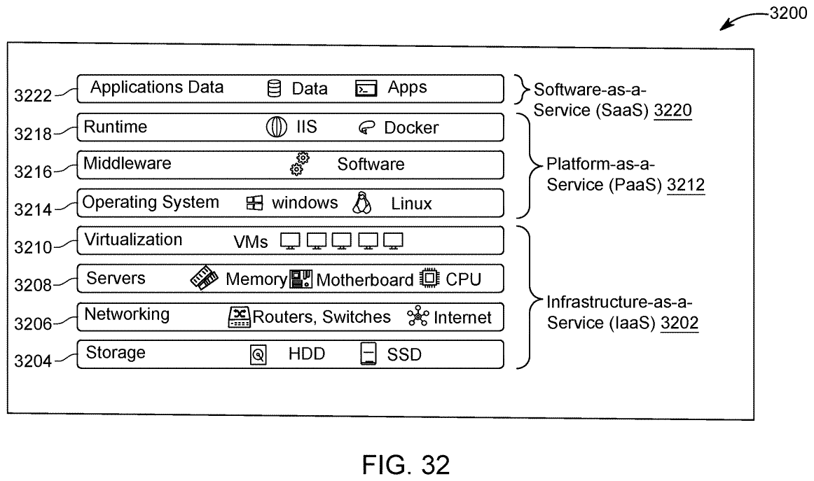

FIG. 32 is a diagram illustrating an exemplary Software-as-a-Service (SaaS) platform suitable for managing healthcare workflows according to an embodiment of the subject matter described herein. Referring to FIG. 32, functionality 3200 can be logically divided into layers. Layers may be ordered from least to greatest abstraction of underlying physical resources. Layers may also be divided into groups based on common features for simplicity when referring or billing functions associated with each group.

Infrastructure 3202 includes storage function 3204, networking function 3206, server function 3208, and virtualization function 3210. Infrastructure functions 3204-3210 may be bundled together and provided to one or more tenants as a service, referred to as Infrastructure-as-a-Service (IaaS). IaaS is made up of a collection of physical and virtualized resources that provide consumers with the basic building blocks needed to run applications and workloads in the cloud.

Storage function 3204 provides storage of data without requiring the user or tenant to be aware of how this data storage is managed. Three types of cloud storage that may be provided by storage function 3204 are block storage, file storage, and object storage. Object storage is the most common mode of storage in the cloud because it is highly distributed (and thus resilient), data can be accessed easily over HTTP, and performance scales linearly as the storage grows.

Networking function 3206 in the cloud is a form of software defined networking in which traditional networking hardware, such as routers and switches, are made available programmatically, typically through APIs.

Server function 3208 refers to various physical hardware resources associated with executing computer-readable code that is not otherwise part of the virtualized network resources in networking function 3206 or storage function 3204. IaaS providers manage large data centers, typically around the world, that contain the servers powering the various layers of abstraction on top of them and that are made available to end users. In most IaaS models, end users do not interact directly with the physical infrastructure (e.g., memory, motherboard, CPU), but it is provided as a service to them.

Virtualization function 3210 provides virtualization of underlying resources via one or more virtual machines (VMs). Virtualization relies on software to simulate hardware functionality and create a virtual computer system. A virtual computer system is known as a “virtual machine” (VM): a tightly isolated software container with an operating system and application inside. Each self-contained VM is completely independent. Putting multiple VMs on a single computer enables several operating systems and applications to run on just one physical server, or “host.” A thin layer of software called a “hypervisor” decouples the virtual machines from the host and dynamically allocates computing resources to each virtual machine as needed. Providers manage hypervisors and end users can then programmatically provision virtual “instances” with desired amounts of compute and memory (and sometimes storage). Most providers offer both CPUs and GPUs for different types of workloads.

Platform 3212 includes operating system function 3214, middleware function 3216, and runtime function 3218. Infrastructure functions 3204-3210 and platform functions 3214-3218 may be bundled together and provided to one or more tenants as a service, referred to as Platform-as-a-Service (PaaS). In the Platform-as-a-Service (PaaS) model, developers rent everything needed to build an application, relying on a cloud provider for development tools, infrastructure, and operating systems.

Operating system function 3214 refers to a PaaS vendor providing and maintaining the operating system that developers use, and the application runs on. For example, Windows and Linux operating systems may be installed in virtual machines and Windows or Linux applications may be run within the operating system.

Middleware function 3216 is software that sits in between user-facing applications and the machine's operating system. For example, middleware may allow software to access input from the keyboard and mouse. Middleware is necessary for running an application, but end users don't interact with it. Relatedly, middleware function 3216 may also include tools that are necessary for software development, such as a source code editor, a debugger, and a compiler. These tools may be offered together as a framework.

Runtime function 3218 is software code that implements portions of a programming language's execution model. A runtime system or runtime environment implements portions of an execution model. Most programming languages have some form of runtime system that provides an environment in which programs run. This environment may address issues including the management of application memory, how the program accesses variables, mechanisms for passing parameters between procedures, interfacing with the operating system, and otherwise. The compiler makes assumptions depending on the specific runtime system to generate correct code. Typically, the runtime system will have some responsibility for setting up and managing the stack and heap, and may include features such as garbage collection, threads or other dynamic features built into the language.

Software 3220 includes applications and data function 3222. Infrastructure functions 3204-3210, platform functions 3214-3218, and software function 3222 may be bundled together and provided to one or more tenants as a service, referred to as Software-as-a-Service (SaaS). Applications and data function 3222 is the application and associated data created and managed by the user. For example, an application programmed by the user for provided certain functionality disclosed herein may be exposed to the end user via a front end interface such as a web browser or dedicated front end client application. Neither the front end user nor the back end developer is required to manage or maintain services provided by platform 3212 and infrastructure 3202. This contrasts with on-site hosting of the same functionality.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for managing healthcare workflows, the method comprising:
obtaining healthcare data associated with a healthcare service from at least one external source;
analyzing the healthcare data;
providing an interface for viewing and managing the healthcare data;
receiving instructions from a user for performing an action; and
performing the action based on the instructions, the healthcare data, and the analysis.

2. The method of claim 1, wherein the healthcare data includes one or more of: an 835, an 837, a 277CA, and a portable document format (PDF) file.

3. The method of claim 2, further comprising using optical character recognition (OCR) on the PDF to convert an image of text into a machine-readable text format.

4. The method of claim 2, wherein the PDF file is associated with one of: a Vonage fax negotiation, an eFax negotiation, and an appeal response.

5. The method of claim 1, wherein analyzing the healthcare data includes extracting key data from the received healthcare data.

6. The method of claim 1, wherein the key data extracted from the received healthcare data includes at least one of: date of birth, invoice number, claim data, identifier number, patient name, phone number, fax number, expiration data, provider name, billed amount, proposed amount, keywords, paid amount, co-insurance, deductible, claim adjustment reason code (CARC), remittance advice remark codes (RARC), place of service (POS), elective versus emergency room (ER) indication, insurance (INS) claim number, and authorization.

7. The method of claim 1, wherein providing an interface for viewing and managing the healthcare data includes displaying a graphical user interface (GUI) to a user.

8. The method of claim 1, wherein performing the action includes at least one of: generating one or more documents associated with an appeal, generating one or more documents associated with a negotiation, generating a phone script, updating a task manager, updating a communication log, generating a report, generating client invoicing.

9. The method of claim 1, wherein performing the action includes at least one of: checking eligibility, receiving documents uploaded by the user, providing a communication log, generating a status report, checking compliance.

10. A system for managing healthcare workflows, the system comprising:
a data storage configured for storing healthcare data associated with a healthcare service; and
a server comprising:
an input module configured for receiving the healthcare data from at least one external source;
an analysis module configured for analyzing the healthcare data stored in the data storage;
an interface module configured for providing an interface for viewing and managing the healthcare data and for receiving instructions from a user for performing an action; and
an application module configured for performing the action based on the instructions, the healthcare data, and the analysis.

11. The method of claim 1, wherein the healthcare data includes one or more of: an 835, an 837, a 277CA, and a portable document format (PDF) file.

12. The method of claim 2, further comprising using optical character recognition (OCR) on the PDF to convert an image of text into a machine-readable text format.

13. The method of claim 2, wherein the PDF file is associated with one of: a Vonage fax negotiation, an eFax negotiation, and an appeal response.

14. The method of claim 1, wherein analyzing the healthcare data includes extracting key data from the received healthcare data.

15. The method of claim 1, wherein the key data extracted from the received healthcare data includes at least one of: date of birth, invoice number, claim data, identifier number, patient name, phone number, fax number, expiration data, provider name, billed amount, proposed amount, keywords, paid amount, co-insurance, deductible, claim adjustment reason code (CARC), remittance advice remark codes (RARC), place of service (POS), elective versus emergency room (ER) indication, insurance (INS) claim number, and authorization.

16. The method of claim 1, wherein providing an interface for viewing and managing the healthcare data includes displaying a graphical user interface (GUI) to a user.

17. The method of claim 1, wherein performing the action includes at least one of: generating one or more documents associated with an appeal, generating one or more documents associated with a negotiation, generating a phone script, updating a task manager, updating a communication log, generating a report, generating client invoicing.

18. The method of claim 1, wherein performing the action includes at least one of: checking eligibility, receiving documents uploaded by the user, providing a communication log, generating a status report, checking compliance.

19. A system for managing healthcare workflows as a service (SaaS), the system comprising:
a computer communicatively coupled to a network, at least one SaaS provider, and at least one SaaS user, and wherein the computer is configured for:
obtaining healthcare data associated with a healthcare service from at least one external source;
analyzing the healthcare data;
providing an interface for viewing and managing the healthcare data;
receiving instructions from a user for performing an action; and
performing the action based on the instructions, the healthcare data, and the analysis.

20. A computer-implemented method comprising instructions stored on a non-transitory computer-readable storage medium and executed on a computing device provided with a hardware processor and a memory for managing healthcare workflows via Software as a Service (SaaS), the method comprising:
obtaining healthcare data associated with a healthcare service from at least one external source;
analyzing the healthcare data;
providing an interface for viewing and managing the healthcare data;
receiving instructions from a user for performing an action; and
performing the action based on the instructions, the healthcare data, and the analysis.
